(19) United States
(12) Patent Application Publication
(10) Pub. No.: US 2008/0207493 A1
(43) Pub. Date: Aug. 28, 2008

(54) Compounds for Use in the Treatment of Obesity

(75) Inventors: Ingrid Pettersson, Frederiksberg (DK); Leif Christensen, Roskide (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 11/908,173

(22) PCT Filed: Mar. 17, 2006
(86) PCT No.: PCT/EP06/60839

§ 371 Date: Nov. 6, 2007

(30) Foreign Application Priority Data
Mar. 17, 2005 (DK) PA 2005 00386

(51) Int. Cl. A61K 38/03; C07K 4/00; A61P 3/04; A61P 1/00

(52) U.S. Cl. 514/9; 530/327; 530/317; 514/15; 514/14

(57) ABSTRACT

The present invention relates to novel peptide compounds which are effective in modulating one or more melanocortin receptor types, to the use of the compounds in therapy, to methods of treatment comprising administration of the compounds to patients in need thereof, and to the use of the compounds in the manufacture of medicaments. The compounds of the invention are of particular interest in relation to the treatment of obesity as well as a variety of diseases or conditions associated with obesity.

### FIELD OF THE INVENTION

The present invention relates to novel peptide compounds which are ligands for one or more melanocortin receptors and which may exert prolonged activity, to the use of the compounds in therapy, to methods of treatment comprising administration of the compounds to patients, and to the use of the compounds in the manufacture of medicaments.

### BACKGROUND OF THE INVENTION

Obesity is a well known risk factor for the development of many very common diseases such as atherosclerosis, hypertension, type 2 diabetes (non-insulin dependent diabetes mellitus (NIDDM)), dyslipidaemia, coronary heart disease, and osteoarthritis and various malignancies. It also causes considerable problems through reduced motility and decreased quality of life. The incidence of obesity and thereby also these diseases is increasing throughout the entire industrialized world. Only a few pharmacological treatments are available to date, namely Sibutramine (Abbot; acting via serotonergic and noradrenaline mechanisms) and Orlistat (Roche Pharm; reducing fat uptake from the gut,). However, due to the important effect of obesity as a risk factor in serious (and even fatal) and common diseases, there is still a need for pharmaceutical compounds useful in the treatment of obesity.

The term obesity implies an excess of adipose tissue. In this context, obesity is best viewed as any degree of excess adiposity that imparts a health risk. The distinction between normal and obese individuals can only be approximated, but the health risk imparted by obesity is probably a continuum with increasing adiposity. However, in the context of the present invention, individuals with a Body Mass Index (BMI=body weight in kilograms divided by the square of the height in meters) above 25 are to be regarded as obese.

Even mild obesity increases the risk for premature death, diabetes, hypertension, atherosclerosis, gallbladder disease and certain types of cancer. In the industrialized western world the prevalence of obesity has increased significantly in the past few decades. Because of the high prevalence of obesity and its health consequences, its treatment should be a high public health priority.

When energy intake exceeds energy expenditure, the excess calories are stored in adipose tissue, and if this net positive balance is prolonged, obesity results, i.e. there are two components to weight balance, and an abnormality on either side (intake or expenditure) can lead to obesity.

Pro-opiomelanocortin (POMC) is the precursor for β-endorphin and melanocortin peptides, including melanocyte stimulating hormone (α-MSH) and adrenocorticotropin (ACTH). POMC is expressed in several peripheral and central tissues including melanocytes, the pituitary, and neurons of the hypothalamus. The POMC precursor is processed differently in different tissues, resulting in the expression of different melanocortin peptides depending on the site of expression. In the anterior lobe of the pituitary, mainly ACTH is produced whereas in the intermediate lobe and the hypothalamic neurons the major peptides are α-MSH, β-MSH, desacetyl-α-MSH and β-endorphin. Several of the melanocortin peptides, including ACTH and α-MSH, have been demonstrated to have appetite-suppressing activity when administered to rats by intracerebroventricular injection [Vergoni et al, European Journal of Pharmacology 179, 347-355 (1990)]. An appetite-suppressing effect is also obtained with the artificial cyclic α-MSH analogue, MT-II.

A family of five melanocortin receptor subtypes has been identified (melanocortin receptor 1-5, also called MC1, MC2, MC3, MC4 and MC5). The MC1, MC2 and MC5 are mainly expressed in peripheral tissues, whereas MC3 and MC4 are mainly centrally expressed; MC3 are, however, also expressed in several peripheral tissues. In addition to being involved in energy homeostasis, MC3 receptors have also been suggested to be involved in several inflammatory diseases. An MC3 agonist could have a positive effect on such diseases, e.g. gouty arthritis. MC5 are mainly peripherally expressed, and have been suggested to be involved in exocrine secretion and in inflammation. MC4 have been shown to be involved in the regulation of body weight and feeding behaviour, as MC4 knock-out mice develop obesity [Huzar et al., Cell 88, 131-141 (1997)]. In addition, the MC4 receptor has been shown to be involved in the regulation of energy expenditure [Fekete et al., Journal of Neuroscience 20, 1550-1558 (2000)]. Furthermore, studies of either ectopic central expression of agouti protein (MC1, MC3 and MC4 antagonist) or over-expression of an endogenously occurring MC3 and MC4 antagonist (agouti gene related protein, AGRP) in mouse brain demonstrated that the over-expression of these two antagonists led to the development of obesity [Kleibig et al., PNAS 92, 4728-4732 (1995)]. Moreover, icv injection of a C-terminal fragment of AGRP increases feeding and antagonizes the inhibitory effect of α-MSH on food intake.

In humans, several cases of families with obesity which is presumably due to frame shift mutations in MC4 have been described [see, e.g., Yeo et al., Nature Genetics 20, 111-112 (1998); Vaisse et al., Nature Genetics 20, 113-114 (1998)].

In conclusion, a MC4 agonist could serve as an anorectic drug or energy expenditure regulating drug and be useful in the treatment of obesity or obesity-related diseases, as well as in the treatment of other diseases, disorders or conditions which may be ameliorated by activation of MC4.

MC4 antagonists may be useful for treatment of cachexia or anorexia, and for treatment of wasting in frail elderly patients. Furthermore, MC4 antagonists may be used for treatment of chronic pain, neuropathy and neurogenic inflammation.

A large number of patent applications disclose various classes of non-peptidic small molecules as melanocortin receptor modulators; examples hereof are WO 03/009850, WO 03/007949 and WO 02/081443.

The use of peptides as melanocortin receptor modulators is disclosed in a number of patent documents, e.g. WO 03/006620, U.S. Pat. No. 5,731,408 and WO 98/27113. Hadley [Pigment Cell Res., 4, 180-185, (1991)] reports a prolonged effect of specific melanotropic peptides conjugated to fatty acids, the prolongation being effected by a transformation of the modulators from being reversibly acting to being irreversibly acting caused by the conjugated fatty acids.

### SUMMARY OF THE INVENTION

The present invention provides specific peptide conjugates having a high modulating effect on one or more melanocortin receptors, i.e. the MC1, MC2, MC3, MC4 or MC5 receptors. In an aspect of the invention, compounds provided by the present invention have a selectivity profile with respect to the receptors. In another aspect of the invention, the selectivity profile is such that the compounds exhibit preference (highest affinity) for the MC4 receptor. In a further aspect of the invention, compounds of the invention exhibit affinity for the MC4 receptor and the MC3 receptor. Particularly interesting peptide conjugates of the invention (compounds of the invention) exhibit a desirable solubility profile under physiological conditions.

Accordingly, the invention relates to compounds of formula I:

R¹—X—X¹—X²—X³—X⁴—X⁵—X⁶—X⁷—X⁸—X⁹—X¹⁰—X¹¹—R² [I]

wherein R¹, which is bonded to an N-terminal NH₂-group, is either absent or represents C₁₋₄alkanoyl or R⁴, which is a protracting group, optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl, C₂₋₈alkenyl or C₂₋₈alkynyl;
wherein the compound of formula I is optionally cyclized from X³ to X¹⁰ via a lactam or a disulfide bridge;
with the proviso that compounds of formula I comprise at least 7 amino acid residues;
and pharmaceutically acceptable salts, prodrugs and solvates thereof.

The invention further relates to the use of compounds of the invention in therapy, to pharmaceutical compositions comprising compounds of the invention, to methods of treatment comprising administration of compounds of the invention to patients in need thereof, and to the use of compounds of the invention in the manufacture of medicaments.

### DEFINITIONS

The use of a prefix of the type “C_{x-y}” preceding the name of a radical (which may also be referred to, e.g., as a substituent, moiety or group), such as in C_{x-y}alkyl (e.g. C₁₋₈alkyl), C_{x-y}alkenyl (e.g. C₂₋₈alkenyl), C_{x-y}alkynyl (e.g. C₂₋₈alkynyl) or C_{x-y}cycloalkyl (e.g. C₃₋₈cycloalkyl), is intended to indicate a radical of the designated type having from x to y carbon atoms. Thus, for example: the prefix “C₁₋₄” indicates a substituent of the type in question having from 1 to 4 (i.e. 1, 2, 3 or 4) carbon atoms, and the prefix “C₁₋₈” indicates a substituent of the type in question having from 1 to 8 (i.e. 1, 2, 3, 4, 5, 6, 7 or 8) carbon atoms. Likewise, for example: the prefix “C₂₋₈” indicates a substituent of the type in question having from 2 to 8 (i.e. 2, 3, 4, 5, 6, 7 or 8) carbon atoms; the prefix “C₈₋₂₂” indicates a substituent of the type in question having from 8 to 22 (i.e. 8, 9, 10, 11, 12, 13, 14, 15, 16, 17, 18, 19, 20, 21 or 22) carbon atoms; the prefix “C₇₋₁₇” indicates a substituent of the type in question having from 7 to 17 (i.e. 7, 8, 9, 10, 11, 12, 13, 14, 15, 16 or 17) carbon atoms; and so on.

The term “alkyl” as used herein refers to a straight-chain, branched and/or cyclic, saturated monovalent hydrocarbon radical, typically an alkyl radical having from one to ten carbon atoms, e.g. from one to eight carbon atoms (i.e. C₁₋₈alkyl). Typical C₁₋₈alkyl groups include, e.g., methyl, ethyl, n-propyl, isopropyl, n-butyl, sec-butyl, isobutyl, tert-butyl, n-pentyl, 2-methylbutyl, 3-methylbutyl, 4-methylpentyl, neopentyl, n-pentyl, n-hexyl, 1,2-dimethylpropyl, 2,2-dimethylpropyl, 1,2,2-trimethylpropyl, cyclopropyl, cyclobutyl, cyclopentyl, cyclohexyl and the like. The term “C₁₋₈alkyl” as used herein also includes secondary C₃₋₈alkyl and tertiary C₄₋₈alkyl. More generally, the term “alkyl” is intended to indicate primary, secondary and tertiary alkyl.

The term “alkenyl” as used herein refers to a straight-chain, branched and/or cyclic, monovalent non-aromatic hydrocarbon radical comprising at least one carbon-carbon double bond, typically an alkenyl radical having from two to ten carbon atoms, e.g. from two to eight carbon atoms (i.e. C₂₋₈alkenyl). Typical C₂₋₈-alkenyl groups include vinyl, 1-propenyl, 2-propenyl, iso-propenyl, 1,3-butadienyl, 1-butenyl, 2-butenyl, 3-butenyl, 2-methyl-1-propenyl, 1-pentenyl, 2-pentenyl, 3-pentenyl, 4-pentenyl, 3-methyl-2-butenyl, 1-hexenyl, 2-hexenyl, 3-hexenyl, 5-hexenyl, 2,4-hexadienyl, 1-, 2- and 3-cyclohexenyl, and the like.

The term “alkynyl” as used herein refers to a straight-chain, branched and/or cyclic, monovalent non-aromatic hydrocarbon radical comprising at least one carbon-carbon triple bond and optionally comprising one or more carbon-carbon double bonds. Such alkynyl radicals in the context of the invention will typically have from two to ten carbon atoms, e.g. from two to eight carbon atoms (i.e. C₂₋₈alkynyl). Examples hereof include ethynyl, 1-propynyl, 2-propynyl, 2-butynyl and 1,3-hexadien-5-ynyl.

The term “alkanoyl” as used herein is intended to indicate a radical of the formula —C(O)—R′, wherein R′ is alkyl as indicated above.

The term “alkenoyl” as used herein is intended to indicate a radical of the formula —C(O)—R″, wherein R″ is alkenyl as indicated above.

The term “alkynoyl” as used herein is intended to indicate a radical of the formula —C(O)—R′″, wherein R′″ is alkynyl as indicated above.

The term “halogen” is intended to indicate a member of the 7^{th} main group of the periodic table of the elements, which includes fluorine, chlorine, bromine and iodine (corresponding to fluoro, chloro, bromo and iodo substituents, respectively).

The term “alkoxy” as used herein is intended to indicate a radical of the formula —OR′, wherein R′ is alkyl as indicated above. Examples hereof include methoxy and ethoxy.

In the present context, the term “aryl” is intended to indicate a carbocyclic aromatic ring radical or a fused aromatic ring system radical wherein at least one of the rings is aromatic. Typical aryl groups include phenyl, biphenylyl, naphthyl, and the like.

In the present context, in a moiety of the type AA(Y), wherein AA denotes an amino acid residue, it is to be understood that the group Y is attached to AA via a functional group in a side chain of the amino acid in question.

When two amino acids are said to be bridged, it is intended to indicate that functional groups in side chains of the two respective amino acids have reacted to form a covalent bond.

In the present context, the term “agonist” is intended to indicate a substance (ligand) that activates the receptor type in question.

In the present context, the term “antagonist” is intended to indicate a substance (ligand) that blocks, neutralizes or counteracts the effect of an agonist.

More specifically, receptor ligands may be classified as follows:

Receptor agonists, which activate the receptor; partial agonists also activate the receptor, but with lower efficacy than full agonists. A partial agonist will behave as a receptor partial antagonist, partially inhibiting the effect of a full agonist

Receptor neutral antagonists, which block the action of an agonist, but do not affect the receptor-constitutive activity.

Receptor inverse agonists, which block the action of an agonist and at the same time attenuate the receptor-constitutive activity. A full inverse agonist will attenuate the receptor-constitutive activity completely; a partial inverse agonist will attenuate the receptor-constitutive activity to a lesser extent.

As used herein the term “antagonist” includes neutral antagonists and partial antagonists, as well as inverse agonists. The term “agonist” includes full agonists as well as partial agonists.

In the present context, the term “pharmaceutically acceptable salt” is intended to indicate a salt which is not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acids, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylenesalicylic, ethanedisulfonic, gluconic, citraconic, aspartic, stearic, palmitic, p-aminobenzoic, glutamic, benzenesulfonic and p-toluenesulfonic acids, EDTA and the like. Further examples of pharmaceutically acceptable inorganic or organic acid addition salts include the pharmaceutically acceptable salts listed in J. Pharm. Sci. (1977) 66, 2, which is incorporated herein by reference. Examples of relevant metal salts include lithium, sodium, potassium and magnesium salts, and the like. Examples of alkylated ammonium salts include methylammonium, dimethylammonium, trimethylammonium, ethylammonium, hydroxyethylammonium, diethylammonium, butylammonium and tetramethylammonium salts, and the like.

As used herein, the term “therapeutically effective amount” of a compound refers to an amount sufficient to cure, alleviate or partially arrest the clinical manifestations of a given disease and/or its complications. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient for the purpose of combating a condition, such as a disease or a disorder. The terms are intended to include the full spectrum of treatments for a given condition from which the patient is suffering, such as administration of the active compound(s) in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the active compound(s) in question to prevent the onset of symptoms or complications. The patient to be treated is preferably a mammal, in particular a human being, but treatment of other animals, such as dogs, cats, cows, horses, sheep, goats or pigs, is within the scope of the invention.

As used herein, the term “solvate” refers to a complex of defined stoichiometry formed between a solute (in casu, a compound according to the present invention) and a solvent. Solvents may include, by way of example, water, ethanol, or acetic acid. A solvate wherein water is the solvent in question is often referred to as a “hydrate”, and such hydrates are thus within the scope of the present invention.

In addition, the following abbreviations have the indicated meanings:

| | |
|---|---|
| Ac | acetyl |
| Abu | 4-aminobutyric acid |
| Ala | alanine |
| Arg | arginine |
| Arg(Pmc) | 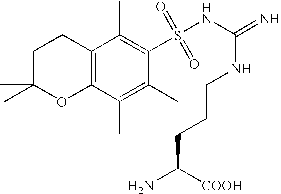 |
| Asn | asparagine |
| Asn(alkyl) | 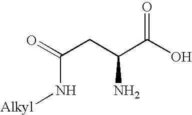 |
| Asn(aryl) | 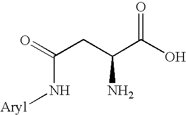 |
| Asp | aspartic acid |
| Boc | tert-butyloxycarbonyl |
| Cys | cysteine |
| Dab | (S)-2,4-diaminobutyric acid |
| Dap | (S)-2,3-diaminopropionic acid |
| D-Phe | 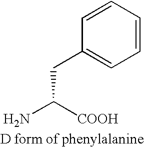 |
| Fmoc | 9-fluorenylmethoxycarbonyl |
| F-Pro | 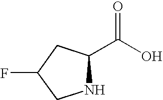 |
| Gly | glycine |
| Gln | glutamine |
| Gln(alkyl) | 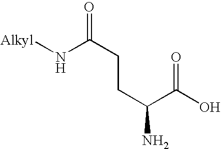 |
| Gln(aryl) | 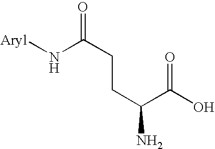 |
| Glu | glutamic acid |
| His | histidine |
| homoArg | 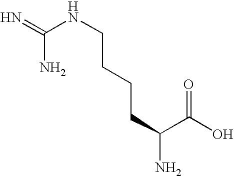 |
| homoCys | 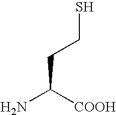 |
| Hyp | 4-hydroxyproline |
| Ile | isoleucine |
| Leu | leucine |
| Lys | lysine |
| Met | methionine |
| Met(O) | 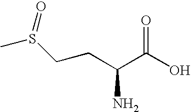 |
| Met(O₂) | 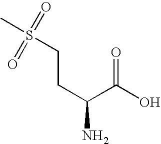 |
| mPEG2000 | methoxy-polyethyleneglycol (average molecular weight of |
| | 2000 Dalton) |
| Mtt | 4-methyltrityl |
| 2-Nal | 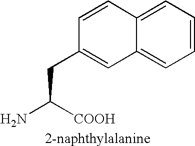 |
| Nle | 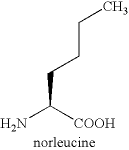 |
| Orn | ornithine |
| Phe | phenylalanine |
| Pro | proline |
| 2-PyAla | 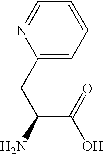 |
| 3-PyAla | 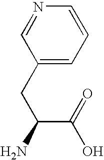 |
| 4-PyAla | 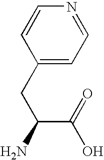 |
| Ser | serine |
| Thr | threonine |
| Tic | (S)-1,2,3,4-tetrahydroisoquinoline-3-carboxylic acid |
| tBu | tert-butyl |
| Trp | tryptophan |
| Val | valine |
| β-Ala | 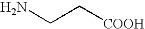 |

Amino acid abbreviations beginning with D- followed by a three letter code, such as D-Ser, D-His and so on, refer to the D-enantiomer of the corresponding amino acid, for example D-serine, D-histidine and so on. In the absence of the letter D preceding a three letter code or amino acid name, as in for example Ser (serine), His (histidine) and so on, it is to be understood that reference is made to the L-enantiomer of the amino acid in question.

### DESCRIPTION OF THE INVENTION

In an embodiment of the invention, R⁴ represents a straight-chain, branched and/or cyclic C₈₋₂₂alkanoyl, C₈₋₂₂alkenoyl or C₈₋₂₂alkynoyl, all of which may optionally be substituted with one or more substituents selected from hydroxy, halogen, carboxyl and aryl, wherein the aryl group in question may optionally be further substituted with one or more substituents selected from hydroxy, halogen and carboxyl;

or R⁴ represents C₇₋₁₇alkyl-C(O)—NH—S(O)₂—(CH₂)₃—C(O)—, wherein the alkyl group in question may be substituted with one or more halogens;
or R⁴ represents R⁵—C(O)—NH—S(O)₂—(CH₂)₃—C(O)—, wherein R⁵ represents 1-(4-benzoyl-phenyl)ethyl;
or R⁴ represents a steroidal group represented by formula II or IIa

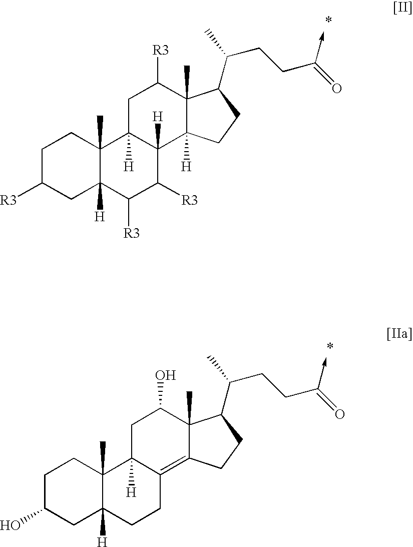

wherein each R³ independently represents hydrogen, hydroxy or (together with the bond via which R³ is attached to the ring carbon atom) ═O;
or R⁴ represents a structure according to formula III, IIIa, IIIb, IV or IVa

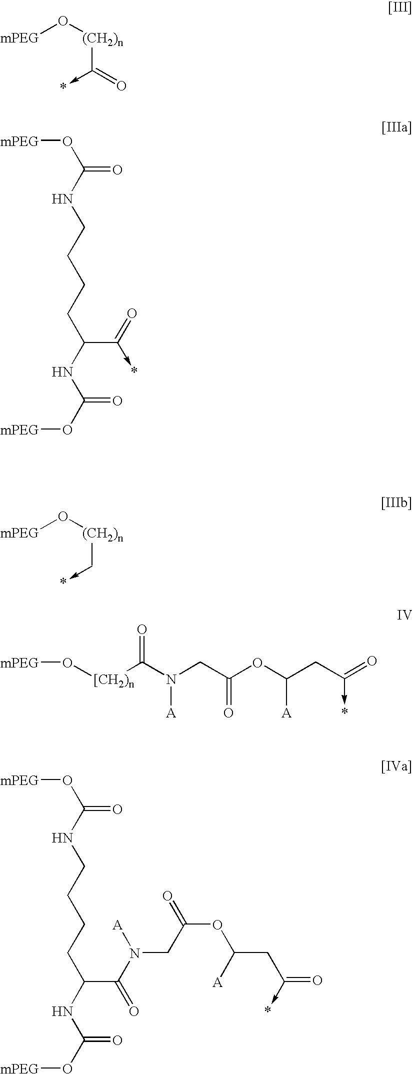

wherein n is 1, 2, or 3;
each mPEG independently represents methoxy-polyethyleneglycol with a molecular weight between about 2 kDa and about 50 kDa; and

each A independently represents hydrogen or C₁₋₄alkyl.

In an embodiment of the invention, X³ represents Lys, Orn, Dab, Dap, Cys, homoCys, Glu, Asp, Gln or Asn;

In an embodiment of the invention X¹⁰ represents Lys, Orn, Dab, Dap, Cys, homoCys, Glu, Asp, Gln or Asp;

In an embodiment of the invention the linker S, if present, represents β-Ala, Glu, Gly-Gln, Gly-Glu, Gly-His, or

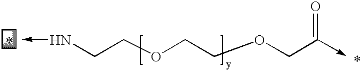

wherein y is 1, 2, 3, 4 or 5.

In an embodiment of the invention there is a bridge between X³ and X¹⁰ rendering the compound of formula I cyclic, either by the presence of a disulfide bridge formed between X³ and X¹⁰ moieties independently selected from Cys and homoCys, or by the presence of a lactam bond formed between a carboxylic acid moiety in the side chain of X³ and an amine moiety in the side chain of X¹⁰, or between a carboxylic acid moiety in the side chain of X¹⁰ and an amine moiety in the side chain of X³.

In an embodiment of the invention, X³ represents Glu or Asp, and X¹⁰ represents Lys, Orn, Dab or Dap. In a further embodiment, X³ represents Glu or Asp, and X¹⁰ represents Lys.

In an embodiment of the invention, R² represents —NH₂.

In an embodiment of the invention, X—X¹—X² is represented by His-Dab-Lys(R⁴) or His-Thr-Lys(R⁴).

In an embodiment of the invention, X—X¹—X² is represented by His-Dab-Lys(S—R⁴) or His-Thr-Lys(S—R⁴).

In an embodiment of the invention, X⁵ represents an amino acid residue selected from Dap, Dab, Orn and Lys. In a further embodiment, X⁵ represents Dab, Orn or Lys.

In an embodiment of the invention, X⁶—X⁷—X⁸—X⁹—X¹⁰ represents Glu-Orn-D-Phe-Arg-Trp-Lys, Glu-Orn-D-Phe-Arg-Trp-Lys, Glu-Orn-D-Phe-Arg-Trp-Lys, Glu-Dab-D-Phe-Arg-Trp-Lys, or Gln-Lys-D-Phe-Arg-Trp-Nle.

In an embodiment of the invention, X—X¹—X²—X³—X⁴—X⁵—X⁶—X⁷—X⁸—X⁹—X¹⁰—X¹¹—R² represents Ac-His-Dab-Lys(hexadecanoyl)-c[Glu-Orn-D-Phe-Arg-Trp-Lys]-NH₂, Ac-His-Dab-Lys(hexadecanoyl)-c[Glu-Lys-D-Phe-Arg-Trp-Lys]-NH₂, Ac-His-Thr-Lys(hexadecanoyl)-c[Glu-Orn-D-Phe-Arg-Trp-Lys]-NH₂, Ac-His-Thr-Lys(hexadecanoyl)-c[Glu-Dab-D-Phe-Arg-Trp-Lys]-NH₂, or Ac-His-Thr-Lys(hexadecanoyl)-Gln-Lys-D-Phe-Arg-Trp-Nle-NH₂.

In an embodiment of the invention, X represents a bond.

In an embodiment of the invention, X² represents Nle.

In an embodiment of the invention, X⁴ represents a bond.

In an embodiment of the invention, X⁵ represents Dab.

In an embodiment of the invention, X⁹ represents a bond.

In an embodiment of the invention, X¹¹ represents a bond.

In an embodiment of the invention, X⁶—X⁷—X⁸—X⁹—X¹⁰ represents D-Phe-Arg-Trp-Lys.

In an embodiment of the invention, X—X¹—X²—X³—X⁴—X⁵—X⁶—X⁷—X⁸—X⁹—X¹⁰—X¹¹—R² represents Ac-His-Ser-Lys(hexadecanoyl)-c[Glu-Dab-D-Phe-Arg-Trp-Lys]-NH₂,

In an embodiment of the invention, X³ represents Glu, and X¹⁰ represents Lys.

In an embodiment of the invention, the compound of formula I is non-cyclic.

In an embodiment of the invention, X represents an amino acid residue.

In an embodiment of the invention, X represents Ser.

In an embodiment of the invention, X¹ represents Lys(—CO—CH₂—CH₂—NH—R⁴).

In an embodiment of the invention, X² represents Tyr-Ser-Nle.

In an embodiment of the invention, X² represents Ser-Nle.

In an embodiment of the invention, X² represents Ser-Tyr-Ser-Nle.

In an embodiment of the invention, X³ represents Glu.

In an embodiment of the invention, X⁵ represents an amino acid residue selected from Dap, Dab, Orn and Lys.

In an embodiment of the invention, X⁹ represents Gly.

In an embodiment of the invention, X¹⁰ represents Lys or Arg.

In an embodiment of the invention, X¹¹ represents Pro-Val.

Specific compounds of the invention include the following, each of which individually constitutes an embodiment of a compound of the invention:

Ac-His-Dab-Lys(4-(hexadecanoylsulfamoyl)butanoyl)-c[Glu-Dab-D-Phe-Arg-Trp-Lys]-NH₂,

### Ac-His-Dab-Lys(hexadecanoyl)-c[Glu-Orn-D-Phe-Arg-Trp-Lys]-NH₂,

### Ac-His-Dab-Lys(hexadecanoyl)-c[Glu-Lys-D-Phe-Arg-Trp-Lys]-NH₂,

### Ac-His-Thr-Lys(hexadecanoyl)-c[Glu-Orn-D-Phe-Arg-Trp-Lys]-NH₂,

### Ac-His-Thr-Lys(hexadecanoyl)-c[Glu-Dab-D-Phe-Arg-Trp-Lys]-NH₂ and

### Ac-His-Thr-Lys(hexadecanoyl)-Gln-Lys-D-Phe-Arg-Trp-Nle-NH₂.

Pharmaceutically acceptable salts, prodrugs and solvates of each of the latter compounds of the invention likewise constitute embodiments of the invention.

### Protractinc Group

One function of the R⁴ substituent is to protract the effect of the compounds, i.e. to prolong the period of time in which they exert a biological activity. A possible protracting effect of a R⁴ substituent may be evaluated in Assay I as described herein (vide infra), by examining the effect of a compound containing the R⁴ substituent to be tested relative to vehicle. The experiment is allowed to continue for a period of time (48 to 72 hours). The time T defines the time-point at which the rats have eaten the same amount of food as the vehicle-treated group during the same time period. T values for the compound containing R⁴ believed to be a protracting group are measured. Groups giving rise to T values ≧24 hours are deemed to be protracting groups. In the context of the present invention, the C₁₋₄alkanoyl group which R¹ may represent is not regarded as a protracting group.

In addition to protracting the effect of a compound, R⁴ may also enhance the potency with which the compound of the present invention modulates the melanocortin receptor compared to a compound wherein R¹ and R⁴ is absent. Such enhanced potency may be measured by testing the compound in a melanocortin assay as described in Assay V herein.

In addition to protracting the effect of a compound, R⁴ may also modify the selectivity profile with respect to the melanocortin receptors compared to a compound in which the R⁴ group in question is absent. Such an altered selectivity profile may be measured by testing the compound in a melanocortin assay as described in Assay II-V herein.

In one embodiment, R⁴ represents a straight-chain, branched and/or cyclic C₈₋₂₂alkanoyl, C₈₋₂₂alkenoyl or C₈₋₂₂alkynoyl, all of which may optionally be substituted with one or more substituents selected from hydroxy, halogen, carboxyl and aryl, wherein the aryl in question may optionally be further substituted with one or more substituents selected from hydroxy, halogen and carboxyl.

In one embodiment, R⁴ represents a straight-chain, branched and/or cyclic C₁₄₋₁₆alkanoyl, C₁₄₋₁₆alkenoyl or C₁₄₋₁₆alkynoyl, all of which may optionally be substituted with one or more substituents selected from hydroxy, halogen, carboxyl and aryl, wherein the aryl in question may optionally be further substituted with one or more substituents selected from hydroxy, halogen and carboxyl.

In one embodiment, R⁴ represents a straight-chain C₁₀₋₂₀alkanoyl, C₁₄₋₁₆alkanoyl or C₈₋₁₇alkanoyl.

In one embodiment, R⁴ represents octanoyl, decanoyl, dodecanoyl, tetradecanoyl, hexadecanoyl, octadecanoyl, 9-carboxy-nonanoyl, 11-carboxy-undecanoyl, 13-carboxy-tridecanoyl, 15-carboxy-pentadecanoyl, 17-carboxy-heptadecanoyl, adamantan-1-yl-acetyl, 4-(hexadecanoyl-sulfamoyl)butanoyl, choloyl, lithocholyl or mPEG2000.

In one embodiment, R⁴ represents a moiety that binds to plasma proteins such as, e.g., albumin. The ability of a compound to bind to albumin may be determined as described in J. Med. Chem. 43, 1986-1992 (2000), which is incorporated herein by reference. In the present context, a compound is defined as binding to albumin if the ratio Ru/Da is greater than 0.05, such as greater than 0.10, e.g. greater than 0.12, or even greater than 0.15.

In an embodiment of the invention, R⁴ represents C₇₋₁₇alkyl-C(O)—NH—S(O)₂—(CH₂)₃—C(O)—, wherein the alkyl in question may be substituted with one or more halogens.

In an embodiment of the invention, R⁴ represents R⁵—C(O)—NH—S(O)₂—(CH₂)₃—C(O)—, wherein R⁵ represents 1-(4-benzoylphenyl)ethyl.

In an embodiment of the invention, R⁴ represents a steroidal group represented by formula II or IIa

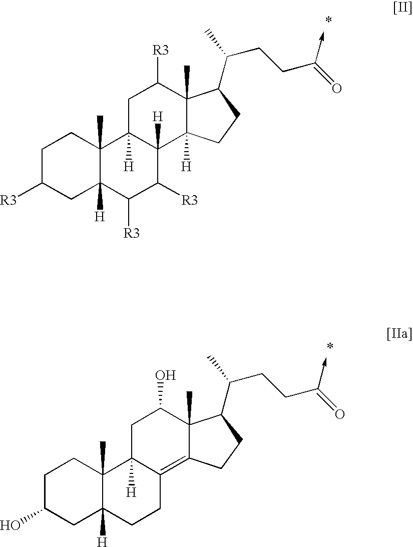

wherein each R³ independently represents hydrogen, hydroxy or (together with the bond via which R³ is attached to the ring carbon atom) ═O.

In an embodiment of the invention, R⁴ represents a structure according to formula III, IIIa, IIIb, IV or IVa

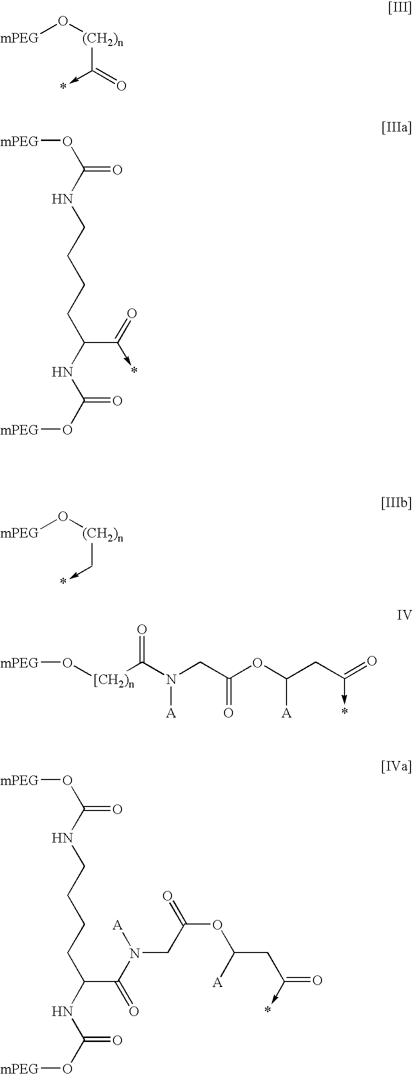

wherein n is 1, 2 or 3;
each mPEG independently represents methoxy-polyethyleneglycol with a molecular weight between about 2 kDa and about 50 kDa; and
each A independently represents hydrogen or C₁₋₄alkyl.

In one aspect of the present invention, the compound of the invention is an agonist of a melanocortin receptor, notably an agonist of MC4. In another aspect of the invention, the compound is a selective agonist of MC4. In this context, selectivity is to be understood in relation to the activity of the compound with respect to MC1, MC3 and/or MC5. If a compound is a significantly more potent as a MC4 agonist than as a MC1, MC3 and/or MC5 agonist, it is deemed to be a selective MC4 agonist. The agonistic potency of a compound with respect to MC1 and MC4 may be determined in receptor binding assays as described below under “Assay IV” (MC1) and “Assay V” (MC4). If a compound is more than 10 times, such as more than 50 times, e.g. more than 100 times more potent with respect to MC4 than with respect to MC1, it is deemed to be a selective MC4 agonist with respect to MC1. The agonistic potency of a compound with respect to MC3, MC4 and MC5 may be determined in functional assays as described in “Assay II” (MC 3 and MC5) and “Assay III” (MC4). If a compound is more than 10 times, such as more than 50 times, e.g. more than 100 times more potent with respect to MC4 than with respect to MC3, it is deemed to be a selective MC4 agonist with respect to MC3. If a compound is more than 10 times, such as more than 50 times, e.g. more than 100 times more potent with respect to MC4 than with respect to MC5, it is deemed to be a selective MC4 agonist with respect to MC5. In a particular aspect, the compound of the present invention is a selective MC4 agonist with respect to MC1, with respect to MC3, with respect to MC5, with respect to MC1 and MC3, with respect to MC1 and MC5, with respect to MC3 and MC5 or with respect to MC1, MC3 and MC5.

In another aspect of the present invention, the compound of the invention is a selective MC4 agonist and a MC3 antagonist. In this context, a compound is deemed to be a selective MC4 agonist and a MC3 antagonist if it is a selective MC4 agonist with respect to MC1 and MC5 as discussed above, and it antagonizes MC3 as determined as described in “Assay II”. In the latter assay, a compound exhibiting an IC₅₀ value of less than 100 nM, such as less than 10 nM, e.g. less than 5 nM, such as less than 1 nM, is deemed to be a MC3 antagonist.

In a further aspect of the present invention, the compound of the present invention is both a selective MC3 agonist and a selective MC4 agonist. In this context, a compound is deemed to be a selective MC3 and MC4 agonist if it is significantly more potent as an agonist towards MC3 and MC4 than as an agonist toward MC1 and MC5. The selectivity of a compound with respect to MC1 and MC3 may be determined by comparing the potency determined for MC1 as described in “Assay IV” with the potency for MC3 determined as described in “Assay II”. If a compound is more than 10 times, such as more than 50 times, e.g. more than 100 times more potent with respect to MC3 than with respect to MC1, it is deemed to be a selective MC3 agonist with respect to MC1. The selectivity of a compound with respect to MC3 and MC5 may be determined by comparing the potency determined as described in “Assay II”. If a compound is more than 10 times, such as more the 50 times, e.g. more than 100 times more potent with respect to MC3 than with respect to MC5, it is deemed to be a selective MC3 agonist with respect to MC5. The MC4 selectivity of a compound with respect to MC3 and MC5 is determined as discussed above.

In an embodiment of the invention, the compound of the invention is an agonist with respect to MC4 and an antagonist with respect to MC3.

Compounds of the present invention modulate melanocortin receptors, and they are therefore believed to be particularly suited for the treatment of diseases or states which can be treated by a modulation of melanocortin receptor activity. In particular, compounds of the present invention are believed to be suited for the treatment of diseases or states via activation of MC4.

In one aspect, the present invention relates to a method of agonizing or activating MC4 in a subject, the method comprising administering to the subject an effective amount of a compound of the present invention (i.e. a compound of formula I).

In another aspect, the invention provides a method of delaying the progression from impaired glucose tolerance (IGT) to type 2 diabetes, the method comprising administering to a patient in need thereof an effective amount of a compound of the present invention.

In a further aspect, the invention provides a method of delaying the progression from type 2 diabetes to insulin-requiring diabetes, the method comprising administering to a patient in need thereof an effective amount of a compound of the present invention.

In an additional aspect, the invention relates to a method of treating obesity or preventing overweight, the method comprising administering to a patient in need thereof an effective amount of a compound of the present invention.

In a still further aspect, the present invention provides a method of regulating appetite, the method comprising administering to a patient in need thereof an effective amount of a compound of the present invention.

Another aspect of the invention relates to a method of inducing satiety, the method comprising administering to a patient in need thereof an effective amount of a compound of the present invention.

A further aspect of the invention relates to a method of preventing weight regain after successfully having lost weight, the method comprising administering to a patient in need thereof an effective amount of a compound of the present invention.

Yet another aspect of the invention relates to a method of increasing energy expenditure, the method comprising administering to a patient in need thereof an effective amount of a compound of the present invention.

Still further aspects of the invention include the following:

a method of treating a disease or state related to overweight or obesity, the method comprising administering to a patient in need thereof an effective amount of a compound of the pre-sent invention;
a method of treating bulimia, the method comprising administering to a patient in need thereof an effective amount of a compound of the present invention;
a method of treating binge-eating, the method comprising administering to a patient in need thereof an effective amount of a compound of the present invention;
a method of treating a disease or state selected from atherosclerosis, hypertension, diabetes, type 2 diabetes, impaired glucose tolerance (IGT), dyslipidemia, coronary heart disease, gallbladder disease, gall stone, osteoarthritis, cancer, sexual dysfunction and risk of premature death, the method comprising administering to a patient in need thereof an effective amount of a compound of the present invention.

In particular, compounds of the present invention may be suited for the treatment of diseases in obese or overweight patients. Accordingly, the present invention also provides a method of treating, in an obese patient, a disease or state selected from type 2 diabetes, impaired glucose tolerance (IGT), dyslipidemia, coronary heart disease, gallbladder disease, gall stone, osteoarthritis, cancer, sexual dysfunction and risk of premature death in obese patients, the method comprising administering to an obese patient in need thereof an effective amount of a compound of the present invention.

Moreover, administration of compounds of the present invention may be advantageous in the treatment of patients, notably obese or overweight patients, who have undergone, or are to undergo, gastric banding and/or gastric surgery.

In addition, MC4 agonists could have a positive effect on insulin sensitivity, on drug abuse by modulating the reward system and on hemorrhagic shock. Furthermore, MC3 and MC4 agonists have antipyretic effects, and both have been suggested to be involved in peripheral nerve regeneration.

In all of the therapeutic methods disclosed above, a compound of the present invention may be administered alone or in combination with one or more (i.e. one or two or three . . . etc.) additional compounds of the present invention. Moreover, a compound of the invention, or a combination of two or more (i.e. two or three or four . . . etc.) compounds of the invention, may be administered in combination with one or more other therapeutically active agents or compounds (i.e. agents or compounds which are not within the scope of the present invention), either sequentially or concomitantly.

A typical dosage of a compound of the invention when employed in accordance with the pre-sent invention is in the range of from about 0.001 to about 100 mg/kg body weight per day, e.g. from about 0.01 to about 50 mg/kg body weight per day, such as from about 0.01 to about 10 mg/kg body weight per day, e.g. from about 0.01 to about 1 mg/kg body weight per day, administered in one or more doses, such as from 1 to 3 doses. The exact dosage will depend upon the frequency and mode of administration, the sex, age, weight and general condition of the subject treated, the nature and severity of the condition treated, any concomitant diseases to be treated and other factors evident to those skilled in the art.

Compounds of the invention may conveniently be formulated in unit dosage form using techniques well known to those skilled in the art. A typical unit dosage form intended for oral administration one or more times per day, such as from one to three times per day, may suitably contain from 0.05 to about 1000 mg, e.g. from about 0.1 to about 500 mg, such as from about 0.5 mg to about 200 mg of a compound of the invention.

In a further aspect, the invention relates to a pharmaceutical composition comprising a compound of the present invention, optionally in combination with one or more additional therapeutically active compounds or substances, together with one or more pharmaceutically acceptable carriers or excipients. The composition may suitably be in unit dosage form comprising from about 0.05 mg to about 1000 mg, such as from about 0.1 mg to about 500 mg, e.g. from about 0.5 mg to about 200 mg, of a compound of the present invention.

The present invention also relates to the use of a compound of the present invention, optionally in combination with one or more additional therapeutically active compounds or substances, in the manufacture of a medicament for the treatment of a disease or condition selected from overweight or obesity, bulimia, binge-eating, atherosclerosis, hypertension, type 2 diabetes, impaired glucose tolerance (IGT), dyslipidemia, coronary heart disease, gallbladder disease, gall stone, osteoarthritis, cancer, sexual dysfunction and risk of premature death.

The invention also relates to the use of a compound of the present invention, optionally in combination with one or more additional therapeutically active compounds or substances, in the manufacture of a medicament effective in: delaying the progression from IGT to type 2 diabetes; delaying the progression from type 2 diabetes to insulin-requiring diabetes; regulating appetite; inducing satiety; preventing weight regain after successfully having lost weight; or increasing energy expenditure.

As described above, compounds of the present invention may be administered or applied in combination with one or more additional therapeutically active compounds or substances. Suitable additional compounds or substances may be selected, for example, from antidiabetic agents, antihyperlipidemic agents, antiobesity agents, antihypertensive agents and agents for the treatment of complications resulting from, or associated with, diabetes.

Suitable antidiabetic agents include: insulin; derivatives or analogues of insulin, including derivatives or analogues exhibiting a profile of protracted or prolonged activity, such as those disclosed in WO 95/07931, WO 97/31022 and WO 2005/012347 (Novo Nordisk A/S), the contents of all of which are incorporated herein by reference; derivatives of GLP-1 (glucagon like peptide-1), such as those disclosed in WO 98/08871 (Novo Nordisk A/S), the contents of which are incorporated herein by reference; derivatives of GLP-1 analogues, such as those disclosed in U.S. Pat. No. 6,458,924 (Knudsen et al.), the contents of which are incorporated herein by reference; and orally active hypoglycemic agents.

Suitable orally active hypoglycemic agents include: imidazolines; sulfonylureas; biguanides; meglitinides; oxadiazolidinediones; thiazolidinediones; insulin sensitizers; α-glucosidase inhibitors; agents acting on the ATP-dependent potassium channel of the pancreatic β-cells, e.g. potassium channel openers such as those disclosed in WO 97/26265, WO 99/03861 and WO 00/37474 (Novo Nordisk A/S), the contents of which are incorporated herein by reference; potassium channel openers such as ormitiglinide; potassium channel blockers such as nateglinide or BTS-67582; glucagon antagonists such as those disclosed in WO 99/01423 and WO 00/39088 (Novo Nordisk A/S and Agouron Pharmaceuticals, Inc.), the contents of which are incorporated herein by reference; GLP-1 agonists such as those disclosed in WO 00/42026 (Novo Nordisk A/S and Agouron Pharmaceuticals, Inc.), the contents of which are incorporated herein by reference; DPP-IV (dipeptidyl peptidase-IV) inhibitors; PTPase (protein tyrosine phosphatase) inhibitors; glucokinase activators, such as those described in WO 2004/002481 (Novo Nordisk), the contents of which are incorporated herein by reference, and in WO 02/08209 (Hoffmann La Roche); inhibitors of hepatic enzymes involved in stimulation of gluconeogenesis and/or glycogenolysis; glucose uptake modulators; GSK-3 (glycogen synthase kinase-3) inhibitors; compounds modifying lipid metabolism, such as antihyperlipidemic agents and antilipidemic agents; compounds lowering food intake; as well as PPAR (peroxisome proliferator-activated receptor) agonists and RXR (retinoid X receptor) agonists such as ALRT-268, LG-1268 or LG-1069.

Other examples of suitable additional therapeutically active substances include insulin or insulin analogues; sulfonylureas, e.g. tolbutamide, chlorpropamide, tolazamide, glibenclamide, glipizide, glimepiride, glicazide or glyburide; biguanides, e.g. metformin; and meglitinides, e.g. repaglinide or senaglinide/nateglinide.

Further examples of suitable additional therapeutically active substances include thiazolidinedione insulin sensitizers, e.g. troglitazone, ciglitazone, pioglitazone, rosiglitazone, isaglitazone, darglitazone, englitazone, CS-011/CI-1037 or T 174, or the compounds disclosed in WO 97/41097 (DRF-2344), WO 97/41119, WO 97/41120, WO 00/41121 and WO 98/45292 (Dr. Reddy's Research Foundation), the contents of all of which are incorporated herein by reference.

Additional examples of suitable additional therapeutically active substances include insulin sensitizers, e.g. GI 262570, YM-440, MCC-555, JTT-501, AR-H039242, KRP-297, GW-409544, CRE-16336, AR-H049020, LY510929, MBX-102, CLX-0940, GW-501516 and the compounds disclosed in WO 99/19313 (NN622/DRF-2725), WO 00/50414, WO 00/63191, WO 00/63192 and WO 00/63193 (Dr. Reddy's Research Foundation), and in WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), the contents of all of which are incorporated herein by reference.

Still further examples of suitable additional therapeutically active substances include:

α-glucosidase inhibitors, e.g. voglibose, emiglitate, miglitol or acarbose;
glycogen phosphorylase inhibitors, e.g. the compounds described in WO 97/09040 (Novo Nordisk A/S);
glucokinase activators; and
agents acting on the ATP-dependent potassium channel of the pancreatic β-cells, e.g. tolbutamide, glibenclamide, glipizide, glicazide, BTS-67582 or repaglinide;

Other suitable additional therapeutically active substances include antihyperlipidemic agents and antilipidemic agents, e.g. cholestyramine, colestipol, clofibrate, gemfibrozil, lovastatin, pravastatin, simvastatin, probucol or dextrothyroxine.

Further agents which are suitable as additional therapeutically active substances include antiobesity agents and appetite-regulating agents. Such substances may be selected from the group consisting of CART (cocaine amphetamine regulated transcript) agonists, NPY (neuropeptide Y) antagonists, Y2 and Y4 receptor agonists, MC3 (melanocortin 3) agonists, MC3 (melanocortin 3) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin reuptake inhibitors (e.g. fluoxetine, seroxat or citalopram), serotonin and norepinephrine reuptake inhibitors, 5HT (serotonin) agonists, bombesin agonists, galanin antagonists, growth hormone, growth factors such as prolactin or placental lactogen, growth hormone releasing compounds (growth hormone secretagogues), ghrelin antagonists, TRH (thyrotropin releasing hormone) agonists, UCP 2 or 3 (uncoupling protein 2 or 3) modulators, chemical uncouplers, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti-related protein) inhibitors, histamine H3 receptor antagonists such as those disclosed in WO 00/42023, WO 00/63208 and WO 00/64884, the contents of all of which are incorporated herein by reference, exendin-4, GLP-1 agonists and ciliary neurotrophic factor.

Further suitable antiobesity agents are bupropion (antidepressant), topiramate (anticonvulsant), ecopipam (dopamine D1/D5 antagonist), naltrexone (opioid antagonist), and peptide YY₃₋₃₆ (Batterham et al, Nature 418, 650-654 (2002)).

An embodiment of a suitable antiobesity agent for use in a method of the invention as an additional therapeutically active substance in combination with a compound of the invention is leptin.

A further embodiment of a suitable antiobesity agent is peptide YY₃₋₃₆.

Additional embodiments of suitable antiobesity agents are serotonin and norepinephrine reuptake inhibitors, e.g. sibutramine.

Other embodiments of suitable antiobesity agents are lipase inhibitors, e.g. orlistat.

Still further embodiments of suitable antiobesity agents are adrenergic CNS stimulating agents, e.g. dexamphetamine, amphetamine, phentermine, mazindol, phendimetrazine, diethylpropion, fenfluramine or dexfenfluramine.

Other examples of suitable additional therapeutically active compounds include antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin.

In certain embodiments of the uses and methods of the present invention, the compound of the present invention may be administered or applied in combination with more than one of the above-mentioned, suitable additional therapeutically active compounds or substances, e.g. in combination with: metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

### Pharmaceutical Compositions

As already mentioned, one aspect of the present invention provides a pharmaceutical composition (formulation) comprising a compound of the present invention. Appropriate embodiments of such formulations will often contain a compound of the invention in a concentration of from 10⁻³ mg/ml to 200 mg/ml, such as, e.g., from 10⁻¹ mg/ml to 100 mg/ml. The pH in such a formulation of the invention will typically be in the range of 2.0 to 10.0. The formulation may further comprise a buffer system, preservative(s), tonicity agent(s), chelating agent(s), stabilizer(s) and/or surfactant(s). In one embodiment of the invention the pharmaceutical formulation is an aqueous formulation, i.e. a formulation comprising water, and the term “aqueous formulation” in the present context may normally be taken to indicate a formulation comprising at least 50% by weight (w/w) of water. Such a formulation is typically a solution or a suspension. An aqueous formulation of the invention in the form of an aqueous solution will normally comprise at least 50% (w/w) of water. Likewise, an aqueous formulation of the invention in the form of an aqueous suspension will normally comprise at least 50% (w/w) of water.

In another embodiment, a pharmaceutical composition (formulation) of the invention may be a freeze-dried (i.e. lyophilized) formulation intended for reconstitution by the physician or the patient via addition of solvents and/or diluents prior to use.

In a further embodiment, a pharmaceutical composition (formulation) of the invention may be a dried formulation (e.g. freeze-dried or spray-dried) ready for use without any prior dissolution.

In a further aspect, the invention relates to a pharmaceutical composition (formulation) comprising an aqueous solution of a compound of the present invention, and a buffer, wherein the compound of the invention is present in a concentration of 0.1-200 mg/ml, such as 0.1-100 mg/ml, and wherein the formulation has a pH of from about 2.0 to about 10.0.

In another embodiment of the invention, the pH of the formulation has a value selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 and 10.0.

In a further embodiment, the buffer in a buffered pharmaceutical composition of the invention may comprise one or more buffer substances selected from the group consisting of sodium acetate, sodium carbonate, citrates, glycylglycine, histidine, glycine, lysine, arginine, sodium dihydrogen phosphate, disodium hydrogen phosphate, sodium phosphate, tris(hydroxymethyl)aminomethane (TRIS), bicine, tricine, malic acid, succinates, maleic acid, fumaric acid, tartaric acid and aspartic acid. Each one of these specific buffers constitutes an alternative embodiment of the invention.

In another embodiment, a pharmaceutical composition of the invention may comprise a pharmaceutically acceptable preservative, e.g. one or more preservatives selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, thiomerosal, bronopol, benzoic acid, imidurea, chlorohexidine, sodium dehydroacetate, chlorocresol, ethyl p-hydroxybenzoate, benzethonium chloride and chlorphenesine (3p-chlorphenoxypropane-1,2-diol). Each one of these specific preservatives constitutes an alternative embodiment of the invention. In a further embodiment of the invention the preservative is present in a concentration from 0.1 mg/ml to 20 mg/ml. In still further embodiments of such a pharmaceutical composition of the invention, the preservative is present in a concentration in the range of 0.1 mg/ml to 5 mg/ml, a concentration in the range of 5 mg/ml to 10 mg/ml, or a concentration in the range of 10 mg/ml to 20 mg/ml. The use of a preservative in pharmaceutical compositions is well known to the skilled person. For convenience, reference is made in this respect to Remington: The Science and Practice of Pharmacy, 20^{th} edition, 2000.

In a further embodiment of the invention the formulation further comprises a tonicity-adjusting agent, i.e. a substance added for the purpose of adjusting the tonicity (osmotic pressure) of a liquid formulation (notably an aqueous formulation) or a reconstituted freeze-dried formulation of the invention to a desired level, normally such that the resulting, final liquid formulation is isotonic or substantially isotonic. Suitable tonicity-adjusting agents may be selected from the group consisting of salts (e.g. sodium chloride), sugars and sugar alcohols (e.g. mannitol), amino acids (e.g. glycine, histidine, arginine, lysine, isoleucine, aspartic acid, tryptophan or threonine), alditols [e.g. glycerol (glycerine), 1,2-propanediol (propyleneglycol), 1,3-propanediol or 1,3-butanediol], polyethyleneglycols (e.g. PEG 400) and mixtures thereof.

Any sugar, such as a mono-, di- or polysaccharide, or a water-soluble glucan, including for example fructose, glucose, mannose, sorbose, xylose, maltose, lactose, sucrose, trehalose, dextran, pullulan, dextrin, cyclodextrin, soluble starch, hydroxyethyl starch or carboxymethylcellulose-sodium, may be used; in one embodiment, sucrose may be employed. Sugar alcohols (polyols derived from mono-, di-, oligo- or polysaccharides) include, for example, mannitol, sorbitol, inositol, galactitol, dulcitol, xylitol, and arabitol. In one embodiment, the sugar alcohol employed is mannitol. Sugars or sugar alcohols mentioned above may be used individually or in combination. There is no fixed limit to the amount used, as long as the sugar or sugar alcohol is soluble in the liquid composition (formulation) and does not adversely effect the stabilizing effects achieved using the methods of the invention. In one embodiment, the concentration of sugar or sugar alcohol is between about 1 mg/ml and about 150 mg/ml.

In further embodiments, the tonicity-adjusting agent is present in a concentration of from 1 mg/ml to 50 mg/ml, such as from 1 mg/ml to 7 mg/ml, from 8 mg/ml to 24 mg/ml, or from 25 mg/ml to 50 mg/ml. A pharmaceutical composition of the invention containing any of the tonicity-adjusting agents specifically mentioned above constitutes an embodiment of the invention. The use of a tonicity-adjusting agent in pharmaceutical compositions is well known to the skilled person. For convenience, reference is made to Remington: The Science and Practice of Pharmacy, 20^{th} edition, 2000.

In a still further embodiment of a pharmaceutical composition (formulation) of the invention, the formulation further comprises a chelating agent. Suitable chelating agents may be selected, for example, from salts of ethylenediaminetetraacetic acid (EDTA), citric acid, and aspartic acid, and mixtures thereof. The concentration of chelating agent will suitably be in the range from 0.1 mg/ml to 5 mg/ml, such as from 0.1 mg/ml to 2 mg/ml or from 2 mg/ml to 5 mg/ml. A pharmaceutical composition of the invention containing any of the chelating agents specifically mentioned above constitutes an embodiment of the invention. The use of a chelating agent in pharmaceutical compositions is well known to the skilled person. For convenience, reference is made to Remington: The Science and Practice of Pharmacy, 20^{th} edition, 2000.

In another embodiment of a pharmaceutical composition (formulation) of the invention, the formulation further comprises a stabilizer. The use of a stabilizer in pharmaceutical compositions is well known to the skilled person. For convenience, reference is made to Remington: The Science and Practice of Pharmacy, 20^{th} edition, 2000.

More particularly, suitable compositions of the invention include stabilized liquid pharmaceutical compositions whose therapeutically active components include an oligo- or polypeptide that in the absence of a stabilizer possibly exhibits aggregate formation during storage in liquid pharmaceutical formulations. By “aggregate formation” is meant the formation of oligomers, which may remain soluble, or large visible aggregates that precipitate from the solution, as the result of a physical interaction between the oligo- or polypeptide molecules. The term “during storage” refers to the fact that a liquid pharmaceutical composition or formulation, once prepared, is not normally administered to a subject immediately. Rather, following preparation, it is packaged for storage, whether in a liquid form, in a frozen state, or in a dried form for later reconstitution into a liquid form or other form suitable for administration to a subject. By “dried form” is meant the product obtained when a liquid pharmaceutical composition or formulation is dried by freeze-drying (i.e., lyophilization; see, for example, Williams and Polli (1984) J. Parenteral Sci. Technol. 38: 48-59), by spray-drying [see, e.g., Masters (1991) in Spray-Drying Handbook (5th edn.; Longman Scientific and Technical, Essex, U.K.), pp. 491-676; Broadhead et al. (1992) Drug Devel. Ind. Pharm. 18: 1169-1206; and Mumenthaler et al. (1994) Pharm. Res. 11: 12-20], or by air-drying [see, e.g., Carpenter and Crowe (1988) Cryobiology 25: 459-470; and Roser (1991) Biopharm. 4: 47-53]. Aggregate formation by an oligo- or polypeptide during storage of a liquid pharmaceutical composition can adversely affect biological activity of that peptide, resulting in loss of therapeutic efficacy of the pharmaceutical composition. Furthermore, aggregate formation may cause other problems, such as blockage of tubing, membranes or pumps when the oligo- or polypeptide-containing pharmaceutical composition is administered using an infusion system.

A pharmaceutical composition of the invention may further comprise an amount of an amino acid base sufficient to decrease aggregate formation by the oligo- or polypeptide during storage of the composition. By “amino acid base” is meant an amino acid, or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids for use in preparing a composition of the invention are those carrying a charged side chain, such as arginine, lysine, aspartic acid and glutamic acid. Any stereoisomer (i.e., L, D, or mixtures thereof) of a particular amino acid (e.g. methionine, histidine, arginine, lysine, isoleucine, aspartic acid, tryptophan or threonine, and mixtures thereof) or combinations of these stereoisomers, may be present in the pharmaceutical compositions of the invention so long as the particular amino acid is present either in its free base form or its salt form. In one embodiment, the L-stereoisomer of an amino acid is used. Compositions of the invention may also be formulated with analogues of these amino acids. By “amino acid analogue” is meant a derivative of a naturally occurring amino acid that brings about the desired effect of decreasing aggregate formation by the oligo- or polypeptide during storage of liquid pharmaceutical compositions of the invention. Suitable arginine analogues include, for example, aminoguanidine, ornithine and N-monoethyl-L-arginine. Suitable methionine analogues include ethionine and buthionine, and suitable cysteine analogues include S-methyl-L-cysteine. As with the amino acids per se, amino acid analogues are incorporated into compositions of the invention in either their free base form or their salt form. In a further embodiment of the invention, the amino acids or amino acid analogues are incorporated in a concentration which is sufficient to prevent or delay aggregation of the oligo- or polypeptide.

In a particular embodiment of the invention, methionine (or another sulfur-containing amino acid or amino acid analogue) may be incorporated in a composition of the invention to inhibit oxidation of methionine residues to methionine sulfoxide when the oligo- or polypeptide acting as the therapeutic agent is a peptide comprising at least one methionine residue susceptible to such oxidation. The term “inhibit” in this context refers to minimization of accumulation of methionine-oxidized species over time. Inhibition of methionine oxidation results in increased retention of the oligo- or polypeptide in its proper molecular form. Any stereoisomer of methionine (L or D) or combinations thereof can be used. The amount to be added should be an amount sufficient to inhibit oxidation of methionine residues such that the amount of methionine sulfoxide is acceptable to regulatory agencies. Typically, this means that no more than from about 10% to about 30% of forms of the oligo- or polypeptide wherein methionine is sulfoxidated are present. In general, this can be achieved by incorporating methionine in the composition such that the ratio of added methionine to methionine residues is in the range from about 1:1 to about 1000:1, such as from about 10:1 to about 100:1.

In a further embodiment of the invention the formulation further comprises a stabilizer selected from high-molecular-weight polymers and low-molecular-weight compounds. Thus, for example, the stabilizer may be selected from substances such as polyethylene glycol (e.g. PEG 3350), polyvinyl alcohol (PVA), polyvinylpyrrolidone, carboxy-/hydroxycellulose and derivatives thereof (e.g. HPC, HPC-SL, HPC-L or HPMC), cyclodextrins, sulfur-containing substances such as monothioglycerol, thioglycolic acid and 2-methylthioethanol, and various salts (e.g. sodium chloride). A pharmaceutical composition of the invention containing any of the stabilizers specifically mentioned above constitutes an embodiment of the invention.

Pharmaceutical compositions of the present invention may also comprise additional stabilizing agents which further enhance stability of a therapeutically active oligo- or polypeptide therein. Stabilizing agents of particular interest in the context of the present invention include, but are not limited to: methionine and EDTA, which protect the peptide against methionine oxidation; and surfactants, notably nonionic surfactants, which protect the polypeptide against aggregation or degradation associated with freeze-thawing or mechanical shearing.

Thus, in a further embodiment of the invention, the pharmaceutical formulation comprises a surfactant, particularly a nonionic surfactant. Examples thereof include ethoxylated castor oil, polyglycolyzed glycerides, acetylated monoglycerides, sorbitan fatty acid esters, polyoxypropylene-polyoxyethylene block polymers (e.g. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (Tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (e.g. phosphatidyl-serine, phosphatidyl-choline, phosphatidylethanolamine, phosphatidyl-inositol, diphosphatidyl-glycerol and sphingomyelin), derivatives of phospholipids (e.g. dipalmitoyl phosphatidic acid) and lysophospholipids (e.g. palmitoyl lysophosphatidyl-L-serine and 1-acyl-sn-glycero-3-phosphate esters of ethanolamine, choline, serine or threonine) and alkyl, alkyl ester and alkyl ether derivatives of lysophosphatidyl and phosphatidylcholines, e.g. lauroyl and myristoyl derivatives of lysophosphatidylcholine, dipalmitoylphosphatidylcholine, and modifications of the polar head group, i.e. cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (e.g. cephalins), glyceroglycolipids (e.g. galactopyranoside), sphingoglycolipids (e.g. ceramides, gangliosides), dodecylphosphocholine, hen egg lysolecithin, fusidic acid derivatives (e.g. sodium taurodihydrofusidate, etc.), long-chain fatty acids (e.g. oleic acid or caprylic acid) and salts thereof, acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no. [577-11-7]), docusate calcium, CAS registry no. [128-49-4]), docusate potassium, CAS registry no. [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-hexadecyl-N,N-dimethyl-3-ammonio-1-propanesulfonate, anionic (alkyl-arylsulfonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyltrimethylammonium bromide, cetylpyridinium chloride), non-ionic surfactants (eg. Dodecyl β-D-glucopyranoside), poloxamines (e.g. Tetronic's), which are tetrafunctional block copolymers derived from sequential addition of propylene oxide and ethylene oxide to ethylenediamine. The surfactant may also be selected from imidazoline derivatives and mixtures thereof. A pharmaceutical composition of the invention containing any of the surfactants specifically mentioned above constitutes an embodiment of the invention.

The use of a surfactant in pharmaceutical compositions is well-known to the skilled person. For convenience, reference is made to Remington: The Science and Practice of Pharmacy, 20^{th} edition, 2000.

In a further embodiment of the invention, the composition (formulation) further comprises a protease inhibitor such as EDTA (ethylenediaminetetraacetic acid) or HCl, but other commercially available protease inhibitors may also be used. The use of a protease inhibitor is particular useful in pharmaceutical compositions comprising zymogens of proteases in order to inhibit autocatalysis.

Additional ingredients may also be present in a pharmaceutical composition (formulation) of the present invention. Such additional ingredients may include, for example, wetting agents, emulsifiers, antioxidants, bulking agents, metal ions, oleaginous vehicles, proteins (e.g. human serum albumin, gelatine or other proteins) and a zwitterionic species (e.g. an amino acid such as betaine, taurine, arginine, glycine, lysine or histidine). Such additional ingredients should, of course, not adversely affect the overall stability of the pharmaceutical formulation of the present invention.

Pharmaceutical compositions containing a compound according to the present invention may be administered to a patient in need of such treatment at one or more of several sites, for example at topical sites (e.g. skin and mucosal sites), at sites which bypass absorption (e.g. via administration in an artery, in a vein or in the heart), and at sites which involve absorption (e.g. in the skin, under the skin, in a muscle or in the abdomen).

Administration of compounds or pharmaceutical compositions according to the invention to patients in need thereof may be via several routes of administration. These include, for example, lingual, sublingual, buccal, in the mouth, oral, in the stomach and intestine, nasal, pulmonary (for example through the bronchioles and alveoli or a combination thereof), epidermal, dermal, transdermal, vaginal, rectal, ocular (for example through the conjunctiva), uretal and parenteral.

Compositions of the present invention may be administered in various dosage forms, for example in the form of solutions, suspensions, emulsions, microemulsions, multiple emulsion, foams, salves, pastes, plasters, ointments, tablets, coated tablets, rinses, capsules (e.g. hard gelatine capsules or soft gelatine capsules), suppositories, rectal capsules, drops, gels, sprays, powder, aerosols, inhalants, eye drops, ophthalmic ointments, ophthalmic rinses, vaginal pessaries, vaginal rings, vaginal ointments, injection solutions, in situ-transforming solutions (for example in situ gelling, in situ setting, in situ precipitating or in situ crystallizing), infusion solutions or implants.

Compositions of the invention may further be compounded in, or bound to, e.g. via covalent, hydrophobic or electrostatic interactions, a drug carrier, drug delivery system or advanced drug delivery system in order to further enhance the stability of the compound of the present invention, increase bioavailability, increase solubility, decrease adverse effects, achieve chronotherapy well known to those skilled in the art, and increase patient compliance, or any combination thereof. Examples of carriers, drug delivery systems and advanced drug delivery systems include, but are not limited to: polymers, for example cellulose and derivatives; polysaccharides, for example dextran and derivatives, starch and derivatives; poly(vinyl alcohol); acrylate and methacrylate polymers; polylactic and polyglycolic acid and block copolymers thereof; polyethylene glycols; carrier proteins, for example albumin; gels, for example thermogelling systems, such as block co-polymeric systems well known to those skilled in the art; micelles; liposomes; microspheres; nanoparticulates; liquid crystals and dispersions thereof; L2 phase and dispersions thereof well known to those skilled in the art of phase behaviour in lipid-water systems; polymeric micelles; multiple emulsions (self-emulsifying, self-microemulsifying); cyclodextrins and derivatives thereof; and dendrimers.

Compositions of the present invention are useful in the formulation of solids, semisolids, powders and solutions for pulmonary administration of a compound of the present invention, using, for example, a metered dose inhaler, dry powder inhaler or a nebulizer, all of which are devices well known to those skilled in the art.

Compositions of the present invention are useful in the formulation of controlled-release, sustained-release, protracted, retarded or slow-release drug delivery systems. Compositions of the invention are thus of value in the formulation of parenteral controlled-release and sustained-release systems well known to those skilled in the art (both types of systems leading to a many-fold reduction in the number of administrations required).

Of particular value are controlled-release and sustained-release systems for subcutaneous administration. Without limiting the scope of the invention, examples of useful controlled release systems and compositions are those containing hydrogels, oleaginous gels, liquid crystals, polymeric micelles, microspheres or nanoparticles,

Methods for producing controlled-release systems useful for compositions of the present invention include, but are not limited to, crystallization, condensation, co-crystallization, precipitation, co-precipitation, emulsification, dispersion, high-pressure homogenisation, encapsulation, spray-drying, microencapsulation, coacervation, phase separation, solvent evaporation to produce microspheres, extrusion and supercritical fluid processes. General reference is made in this context to Handbook of Pharmaceutical Controlled Release (Wise, D. L., ed. Marcel Dekker, New York, 2000), and Drugs and the Pharmaceutical Sciences, vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, for example a syringe in the form of a pen device. Alternatively, parenteral administration may be performed by means of an infusion pump. A further option is administration of a composition of the invention which is a liquid (typically aqueous) solution or suspension in the form of a nasal or pulmonary spray. As a still further option, a pharmaceutical composition of the invention can be adapted to trans-dermal administration (e.g. by needle-free injection or via a patch, such as an iontophoretic patch) or transmucosal (e.g. buccal) administration.

A compound or composition of the invention may be administered via the pulmonary route in a vehicle, as a solution, suspension or dry powder using any of a number of known types of devices suitable for pulmonary drug delivery. Examples of these include, but are not limited to, the three general types of aerosol-generating devices for pulmonary drug delivery, and may include jet or ultrasonic nebulizers, metered-dose inhalers, or dry powder inhalers [cf. Yu J., Chien Y. W., Pulmonary drug delivery: Physiologic and mechanistic aspects, Crit. Rev. Ther. Drug Carr. Sys. 14(4) 395-453 (1997)].

Based on standardised testing methodology, the aerodynamic diameter (dₐ) of a particle is defined as the geometric equivalent diameter of a reference standard spherical particle of unit density (1 g/cm³). In the simplest case, for spherical particles, dₐ is related to a reference diameter (d) as a function of the square root of the density ratio as described by:

$${d}_{a} = \sqrt{\frac{ρ}{{ρ}_{a}}} d$$

Modifications to this relationship occur for non-spherical particles [see, e.g., Edwards D. A., Ben-Jebria A., Langer R., Recent advances in pulmonary drug delivery using large, porous inhaled particles, J. Appl. Physiol. 84(2) 379-385 (1998)]. The terms “MMAD” and “MMEAD” are well described and well known (see Edwards D. A. et al., loc. cit.), and represent a measure of the median value of an aerodynamic particle size distribution. Mass median aerodynamic diameter (MMAD) and mass median effective aerodynamic diameter (MMEAD), used interchangeably, are statistical parameters, and they describe empirically the size of aerosol particles in relation to their potential to deposit in the lungs, independent of actual shape, size or density (see Edwards D. A. et al., loc. cit.). MMAD is normally calculated from measurements made with an impactor, an instrument that measures the particle inertial behaviour in air.

In a further embodiment, a composition of the invention may be aerosolized by any known aerosolization methodology, such as nebulisation, to achieve a MMAD of aerosol particles less than 10 μm, more preferably from 1 to 5 μm, such as from 1 to 3 μm. An appropriate particle size is based on the most effective size for delivery of drug to the deep lung, where protein is optimally absorbed (see Edwards D. A. et al., loc. cit.).

Deep lung deposition of pulmonary compositions comprising a compound of the invention may be further optimized by using modified inhalation techniques, including—but not limited to—slow inhalation flow (e.g. 30 L/min), breath holding and timing of actuation.

The term “stabilized formulation” refers to a formulation (composition) with increased physical stability, increased chemical stability or increased physical and chemical stability. The term “physical stability” in the context of a formulation containing an oligo- or polypeptide refers to the tendency of the peptide to form biologically inactive and/or insoluble aggregates as a result of exposure to thermo-mechanical stresses and/or interaction with interfaces and surfaces that are destabilizing, such as hydrophobic surfaces and interfaces. Physical stability of aqueous protein formulations is evaluated by means of visual inspection and/or turbidity measurements after exposing the formulation, filled in suitable containers (e.g. cartridges or vials), to mechanical/physical stress (e.g. agitation) at different temperatures for various time periods. Visual inspection of formulations is performed in a sharp focused light with a dark background. The turbidity of a formulation is characterized by a visual score ranking the degree of turbidity, for instance on a scale from 0 to 3 (in that a formulation showing no turbidity corresponds to a visual score 0, whilst a formulation showing visual turbidity in daylight corresponds to visual score 3). A formulation is normally classified physically unstable with respect to aggregation when it shows visual turbidity in daylight. Alternatively, the turbidity of a formulation can be evaluated by simple turbidity measurements well-known to the skilled person. Physical stability of aqueous oligo- or polypeptide formulations can also be evaluated by using a spectroscopic agent or probe of the conformational status of the peptide. The probe is preferably a small molecule that preferentially binds to a non-native conformer of the oligo- or polypeptide. One example of a small-molecular spectroscopic probe of this type is Thioflavin T. Thioflavin T is a fluorescent dye that has been widely used for the detection of amyloid fibrils. In the presence of fibrils, and possibly also other configurations, Thioflavin T gives rise to a new excitation maximum at about 450 nm, and enhanced emission at about 482 nm when bound to a fibril form. Unbound Thioflavin T is essentially non-fluorescent at the wavelengths in question.

Other small molecules can be used as probes of the changes in peptide structure from native to non-native states. Examples are the “hydrophobic patch” probes that bind preferentially to exposed hydrophobic patches of a polypeptide. The hydrophobic patches are generally buried within the tertiary structure of a polypeptide in its native state, but become exposed as it begins to unfold or denature. Examples of such small-molecular, spectroscopic probes are aromatic, hydrophobic dyes, such as anthracene, acridine, phenanthroline and the like. Other spectroscopic probes are metal complexes of amino acids, such as cobalt complexes of hydrophobic amino acids, e.g. phenylalanine, leucine, isoleucine, methionine, valine, or the like.

The term “chemical stability” of a pharmaceutical formulation as used herein refers to chemical covalent changes in oligo- or polypeptide structure leading to formation of chemical degradation products with potentially lower biological potency and/or potentially increased immunogenicity compared to the original molecule. Various chemical degradation products can be formed depending on the type and nature of the starting molecule and the environment to which it is exposed. Elimination of chemical degradation can most probably not be completely avoided, and gradually increasing amounts of chemical degradation products may often be seen during storage and use of oligo- or polypeptide formulations, as is well known to the person skilled in the art. A commonly encountered degradation process is deamidation, a process in which the side-chain amide group in glutaminyl or asparaginyl residues is hydrolyzed to form a free carboxylic acid. Other degradation pathways involve formation of higher molecular weight transformation products wherein two or more molecules of the starting substance are covalently bound to each other through transamidation and/or disulfide interactions, leading to formation of covalently bound dimer, oligomer or polymer degradation products (see, e.g., Stability of Protein Pharmaceuticals, Ahern. T. J. & Manning M. C., Plenum Press, New York 1992). Oxidation (of for instance methionine residues) may be mentioned as another variant of chemical degradation. The chemical stability of a formulation may be evaluated by measuring the amounts of chemical degradation products at various time-points after exposure to different environmental conditions (in that the formation of degradation products can often be accelerated by, e.g., increasing temperature). The amount of each individual degradation product is often determined by separation of the degradation products on the basis of molecular size and/or charge using various chromatographic techniques (e.g. SEC-HPLC and/or RP-HPLC).

Hence, as outlined above, a “stabilized formulation” refers to a formulation with increased physical stability, increased chemical stability, or increased physical and chemical stability. In general, a pharmaceutical composition (formulation) must be stable during use and storage (in compliance with recommended use and storage conditions) until the expiry date is reached.

A pharmaceutical composition (formulation) of the invention should preferably be stable for more than 2 weeks of usage and for more than two years of storage, more preferably for more than 4 weeks of usage and for more than two years of storage, desirably for more than 4 weeks of usage and for more than 3 years of storage, and most preferably for more than 6 weeks of usage and for more than 3 years of storage.

Compounds according to the present invention may be administered alone or in combination with pharmaceutically acceptable carriers or excipients, in either single or multiple doses. The pharmaceutical compositions according to the present invention may be formulated with pharmaceutically acceptable carriers or diluents as well as any other known adjuvants and excipients in accordance with conventional techniques, such as those disclosed in Remington: The Science and Practice of Pharmacy, 20^{th} Edition, Gennaro, Ed., Mack Publishing Co., Easton, Pa., 2000.

The pharmaceutical compositions may be specifically formulated for administration by any suitable route, such as the oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route, the parenteral or sublingual routes often being preferable. It will be appreciated that the preferred route will depend on the general condition and age of the subject to be treated, the nature of the condition to be treated, and the nature of the active substance (compound of the invention) chosen.

Pharmaceutical compositions for oral administration include solid dosage forms such as hard or soft capsules, tablets, troches, dragees, pills, lozenges, powders or granules. Where appropriate, they may be prepared with coatings, such as enteric coatings, or they may be formulated so as to provide controlled release of the active ingredient, such as sustained or prolonged release, according to methods well known in the art.

Liquid dosage forms for oral administration include solutions, emulsions, aqueous or oily suspensions, syrups and elixirs.

Pharmaceutical compositions for parenteral administration include sterile aqueous and non-aqueous injectable solutions, dispersions, suspensions and emulsions, as well as sterile powders to be reconstituted in sterile injectable solutions or dispersions prior to use. Depot injectable formulations are also within the scope of the present invention.

Other suitable administration forms include suppositories, sprays, ointments, crèmes, gels, inhalants, dermal patches, implants, etc.

As already indicated above, pharmaceutical compositions (formulations) of the invention may conveniently be presented in unit dosage form by methods known to those skilled in the art, and a typical unit dosage form for oral administration one or more times per day, such as from one to three times per day, may suitably contain from about 0.05 to about 1000 mg, such as from about 0.1 to about 500 mg, e.g. from about 0.5 mg to about 200 mg, of a compound of the invention. For parenteral routes such as intravenous, intrathecal, intramuscular and similar administration routes, suitable doses will often be of the order of about half the dose employed for oral administration.

For parenteral administration, a solution of a compound according to the present invention in a suitable liquid medium, e.g. sterile aqueous solution, aqueous propyleneglycol, sesame oil or peanut oil, may be employed. Aqueous solutions should be suitably buffered if necessary, and the liquid diluent rendered isotonic by incorporation of sufficient tonicity-adjusting agent (e.g. saline or a sugar such as glucose). Aqueous solutions are particularly suitable for intravenous, intramuscular, subcutaneous and intraperitoneal administration. The sterile aqueous media employed are all readily available by standard techniques known to those skilled in the art.

In general, suitable pharmaceutical carriers include inert solid diluents or fillers, sterile aqueous solution and various organic solvents. Examples of solid carriers are lactose, terra alba, sucrose, cyclodextrin, talc, gelatin, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Moreover, the carrier or diluent may include any sustained release material known in the art, such as glyceryl monostearate or glyceryl distearate, alone or mixed with a wax.

Orally available formulations may, for example, be in the form of powder or granules, a solution or suspension in an aqueous or non-aqueous liquid, or an oil-in-water or water-in-oil liquid emulsion. Compositions intended for oral use may be prepared according to any known method, and such compositions may contain one or more agents selected from sweetening agents, flavouring agents, colouring agents and preserving agents in order to provide pharmaceutically elegant and palatable preparations. Tablets may contain the active ingredient in admixture with non-toxic pharmaceutically acceptable excipients which are suitable for the manufacture of tablets. These excipients may, for example, be inert diluents, such as calcium carbonate, sodium carbonate, lactose, calcium phosphate or sodium phosphate; granulating and disintegrating agents, such as corn starch or alginic acid; binding agents, e.g. starch, gelatine or acacia; and lubricating agents, such as magnesium stearate, stearic acid or talc. Tablets may be uncoated, or they may be coated by known techniques to delay disintegration and absorption in the gastrointestinal tract and thereby provide a sustained action over a longer period. For example, a time-delay material such as glyceryl monostearate or glyceryl distearate may be employed. Tablets may also be coated by techniques described in U.S. Pat. No. 4,356,108, U.S. Pat. No. 4,166,452 and U.S. Pat. No. 4,265,874, the contents of which are incorporated herein by reference, to form osmotic therapeutic tablets for controlled release.

Formulations for oral use may also be presented as hard gelatine capsules in which the active substance is mixed with an inert solid diluent, e.g. calcium carbonate, calcium phosphate or kaolin, or as soft gelatine capsules wherein the active substance is mixed with water or an oil medium, such as peanut oil, liquid paraffin or olive oil.

Aqueous suspensions may contain the active substance in admixture with excipients suitable for the manufacture of aqueous suspensions. Such excipients include: suspending agents, e.g. sodium carboxymethylcellulose, methylcellulose, hydroxypropylmethylcellulose, sodium alginate, polyvinylpyrrolidone, gum tragacanth or gum acacia; dispersing or wetting agents, e.g. a naturally occurring phosphatide such as lecithin, or condensation products of an alkylene oxide with fatty acids, e.g. polyoxyethylene stearate, or condensation products of ethylene oxide with long-chain aliphatic alcohols, e.g. heptadecaethyleneoxycetanol, or condensation products of ethylene oxide with partial esters derived from fatty acids and a hexitol such as polyoxyethylene sorbitol mono-oleate, or condensation products of ethylene oxide with partial esters derived from fatty acids and hexitol anhydrides, e.g. polyethylene sorbitan mono-oleate. Aqueous suspensions may also contain one or more colouring agents, one or more flavouring agents, and/or one or more sweetening agents, e.g. a sweetening agent such as sucrose or saccharin.

Oily suspensions may be formulated by suspending the active substance in a vegetable oil, e.g. arachis oil, olive oil, sesame oil or coconut oil, or in a mineral oil such as a liquid paraffin. Oily suspensions may contain a thickening agent, e.g. beeswax, hard paraffin or cetyl alcohol. Sweetening agents, such as those set forth above, and flavoring agents may be added to provide a palatable oral preparation. These compositions may be preserved by the addition of an anti-oxidant, such as ascorbic acid.

Dispersible powders and granules suitable for preparation of an aqueous suspension by the addition of water provide the active substance in admixture with a dispersing or wetting agent, suspending agent and normally one or more preservatives. Suitable dispersing or wetting agents and suspending agents include those already mentioned above. Additional excipients, e.g. sweetening, flavoring and/or colouring agents, may also be present. Pharmaceutical compositions according to the present invention may also be in the form of oil-in-water emulsions. The oily phase may be a vegetable oil, e.g. olive oil or arachis oil, or a mineral oil, e.g. a liquid paraffin, or a mixture thereof. Suitable emulsifying agents may be naturally-occurring gums, e.g. gum acacia or gum tragacanth, naturally-occurring phosphatides, e.g. soy bean, lecithin, and esters or partial esters derived from fatty acids and hexitol anhydrides, e.g. sorbitan monooleate, as well as condensation products of such partial esters with ethylene oxide, e.g. polyoxyethylene sorbitan monooleate. The emulsions may also contain sweetening and/or flavoring agents.

Syrups and elixirs may be formulated with sweetening agents, e.g. glycerol, propyleneglycol, sorbitol or sucrose. Such formulations may also contain a demulcent, a preservative, a flavoring agent and/or a coloring agent.

Pharmaceutical compositions of the invention may also be in the form of suppositories for rectal administration of compounds of the present invention. Such suppositories may be pre-pared by mixing the active substance with a suitable non-irritating excipient which is solid at ordinary temperatures, but which is liquid at rectal temperature and will thus melt in the rectum to release the drug. Such materials include, for example, cocoa butter and polyethyleneglycols, for example.

For topical use, creams, ointments, jellies, solutions or suspensions, etc., containing a compound of the present invention are or relevance. In the present context, topical application includes the use of mouth washes and gargles.

Compounds according to the present invention may also be administered in the form of liposome delivery systems, such as small unilamellar vesicles, large unilamellar vesicles or multilamellar vesicles. Liposomes may be formed from a variety of phospholipids, such as cholesterol, stearylamine or phosphatidylcholines.

All references, including publications, patent applications and patents, cited herein are hereby incorporated by reference in their entirety and to the same extent as if each reference were individually and specifically indicated to be incorporated by reference and were set forth in its entirety herein (to the maximum extent permitted by law).

Headings and sub-headings are used herein for convenience only, and should not be construed as limiting the invention in any way.

The use of any and all examples, or exemplary language (including “for instance”, “for example”, “e.g.” and “such as”) in the present specification is intended merely to better illuminate the invention, and does not pose a limitation on the scope of the invention unless otherwise indicated. No language in the specification should be construed as indicating any non-claimed element as being essential to the practice of the invention.

The citation and incorporation of patent documents herein is done for convenience only, and does not reflect any view of the validity, patentability and/or enforceability of such patent documents.

The present invention includes all modifications and equivalents of the subject matter recited in the claims appended hereto, as permitted by applicable law.

### EXAMPLES

### List of Abbreviations Employed

AcOEt ethyl acetate
DCM dichloromethane
DIC diisopropylcarbodiimide
DIPEA ethyldiisopropylamine

### DMAP 4-N,N-dimethylaminopyridine

### DMEM Dulbecco's Modified Eagle's Medium

### DMF N,N-dimethylformamide

DMSO dimethyl sulfoxide
EGTA 1,2-di(2-aminoethoxy)ethane-N,N,N′,N′-tetraacetic acid
FCS fetal calf serum
Fmoc 9-fluorenylmethyloxycarbonyl
HBTU 2-(1H-Benzotriazole-1-yl)-1,1,3,3-tetramethyluronium hexafluorophosphate
HEPES 2-[4-(2-hydroxyethyl)-piperazin-1-yl]-ethanesulfonic acid
HOAt 1-hydroxy-7-azabenzotriazole
HOBt 1-hydroxybenzotriazole
HSA human serum albumin
MeCN acetonitrile
α-MSH α-form of melanocyte-stimulating hormone
Mtt 4-methyltrityl
MTX methotrexate

### NMP N-methylpyrrolidone

Pbf 2,2,4,6,7-pentamethyldihydro-benzofuran-5-sulfonyl
PBS phosphate-buffered saline
PEI polyethyleneimine
PyBop (benzotriazol-1-yloxy)tripyrrolidino-phosphonium hexafluorophosphate
TFA trifluoroacetic acid

All compounds of the present invention can be synthesized by those skilled in the art using standard coupling and deprotection steps. A description of all necessary tools and synthetic methods including standard abbreviations for peptide synthesis can be found in “The Fine Art Of Solid Phase Synthesis”, 2002/3 Catalogue, Novabiochem.

A typical example of a synthesis of a compound of the invention which includes a cyclization step is as follows:

### Example A

### Ac-His-Dab-Lys(4-(Hexadecanoylsulfamoyl)butanoyl)-c[Glu-Dab-D-PheArg-Trp-Lys]-NH₂

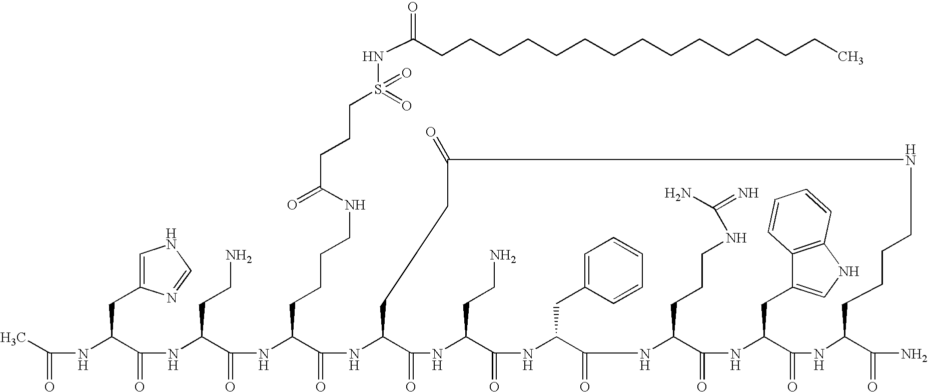

The protected peptidyl resin Fmoc-Glu(2-phenylisopropyloxy)-Dab-DPhe-Arg(Pbf)-Trp(Boc)Lys(Mtt)-NH2 was synthesized according to the Fmoc strategy on a MultiSyntech semiautomatic synthesizer equipped with one reactor in 3.25 mmol scale on a commercial Rink amide resin. The protocol employed (1) 0.45 M HBTU in DMF, (2) 2 M DIPEA in NMP and (3) 0.5 M solution of the protected Fmoc-amino acid solvated in a 0.5 M solution of HOBt/HoAt (1:1) in NMP. The amino acids were pre-activated by mixing the three solutions (18 ml (1), 14 ml (2) and 18 ml (3)) and agitating the solution for 10 min. Reactions were run for 2 h with the exception of the coupling of the first Fmoc-Lys(Mtt), which was repeated once. The resin was washed 3 times with 60 ml NMP before removal of the Fmoc protection group. Fmoc-removal was performed by treating the resin with 50 ml 20% piperidine in NMP twice for 5 and 15 min, respectively, followed by 6×60 ml NMP wash. The Fmoc-removal and -coupling steps were repeated until the desired sequence was obtained.

The peptidyl resin was washed with 3×60 ml DCM and treated for 6×10 min. with 50 ml 2% trifluoroacetic acid (TFA), 2.5% TES in DCM, under regular mixing. The resin was washed with 5×50 ml DCM, with NMP with 5% DIPEA, and with NMP. The peptide was cyclized using HOBt (13 mmol), PyBop (13 mmol) and DIPEA (39 mmol) in DMF (50 ml) with regular mixing for 16 h. The resin was washed with 4×60 ml NMP and 6×60 ml DCM.

Fmoc-Lys(Mtt)-OH was coupled according to the above protocol. Removal of the Mtt group was done as described above.

Attachment of the 4-(hexadecanoylsulfamoyl)butyric acid was performed by adding a 0.5 M solution of the acid and DIC (1:1) in neat DMF to the resin. After 10 minutes, 3 equiv. of DIPEA were added and the reaction was allowed to proceed overnight at room temperature.

The remaining two amino acids, Dab and His, were attached according to the procedure described above.

The peptide was cleaved from the resin by stirring for 120 minutes at room temperature with 35 ml TFA with 2.5% water and 2.5% TES. The cleavage mixture was filtered, and the resin was washed with a further 10 ml of TFA. The crude peptide was precipitated from the combined filtrate by addition of 400 ml of diethyl ether, and isolated by centrifugation. The precipitate was washed twice with diethyl ether.

The crude cyclic peptide was purified by preparative RP-HPLC in accordance with the following procedure:

The crude peptide is dissolved in water/acetonitrile (65:35) (100 ml) adjusted to pH 7.5 with NH₄OH and purified by semipreparative HPLC on a 25 mm×250 mm column packed with 7μ C-18 silica. The column is eluted with a gradient of 50 to 70% acetonitrile against 0.1% TFA/water at 10 ml/min at a temperature of 40° C. for 47 minutes. The peptide-containing fractions are collected, diluted with 3 volumes of water and lyophilized.

The final product obtained is characterized by RP-HPLC/ion spray mass spectrometry (LCMS) (retention time and molecular mass) and by analytical RP-HPLC (retention time).

Peptide concentration is calculated on the basis of the absorbance of the Trp residue at 280 nm in a 1-cm cell, as follows:

mg peptide/ml=(absorbance×dilution factor×molecular weight)/(number of Trp residues×5560 AU/mmol/ml)

The RP-HPLC analysis is performed on a Vydac 218TP54 4.6 mm×250 mm 5μ C-18 silica column (The Separations Group, Hesperia) with UV detection at 214 nm. The column is equilibrated with 0.1% TFA/H₂O and eluted with a gradient of 0 to 90% MeCN against 0.1% TFA/water for 50 min at 42° C., with a flow rate of 0.5 ml/min.

The LC-MS analysis is performed on a XTerra MS C₁₈ 5 μl 3.0×50 mm column (Waters, Milford MA, USA) which is eluted at 1 ml/min at room temperature. The HPLC system employed is equipped with a Sciex API 150 mass spectrometer scanning from 200-1500 amu every 2 seconds of the run.

### Gradient:

| | Total Time | Flow Rate | CAN 0.08% | MQ 0.1% |
|---|---|---|---|---|
| Step | (min) | (μl/min) | TFA (%) | TFA (%) |
| 0 | 1.00 | 1500 | 5.0 | 95.0 |
| 1 | 3.00 | 1500 | 20.0 | 80.0 |
| 2 | 16.00 | 1500 | 50.0 | 50.0 |
| 3 | 18.00 | 1500 | 90.0 | 10.0 |
| 4 | 18.10 | 1500 | 5.0 | 95.0 |
| 5 | 20.00 | 1500 | 5.0 | 95.0 |
| 6 | 22.00 | 0 | 0.0 | 100.0 |

### Preparation of 4-(hexadecanoylsulfamoyl)butyric Acid Employed in the Synthesis of the Compound Above

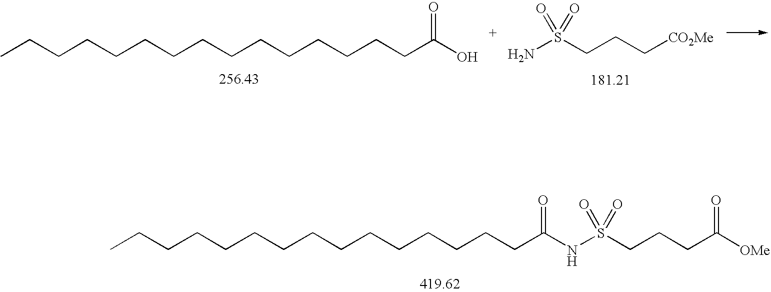

### Step 1:

To a suspension of palmitic acid (1.67 g, 6.51 mmol) in toluene (6.0 ml) was added oxalyl chloride (0.56 ml, 6.53 mmol). After 45 min the resulting clear solution was added to a flask containing 4-sulfamoylbutyric acid methyl ester (0.91 g, 5.02 mmol), and the mixture was diluted with DCM (5.0 ml). To this mixture was added 4-dimethylaminopyridine (DMAP, 1.90 g, 15.5 mmol) in small portions. The mixture was stirred at room temperature for 19 h. A mixture of water (100 ml) and 1N HCl (20 ml) was added, followed by extraction with AcOEt/DCM, washing of the combined extracts with brine, drying (MgSO4), and concentration under reduced pressure. The resulting solid (2.26 g) was recrystallized from hot AcOEt (approx. 10 ml), giving 1.59 g (76%) of the methyl ester as an almost colorless solid, melting point (m.p.): 100-103° C.

¹H NMR (DMSO-d₆): δ 0.84 (m, 3H), 1.23 (br s, 24H), 1.49 (m, 2H), 1.88 (m, 2H), 2.24 (t, J=7 Hz, 2H), 2.49 (t, J=7 Hz, 2H), 3.38 (m, 2H), 3.59 (s, 3H), 11.58 (s, 1H).

### Step 2: Saponification

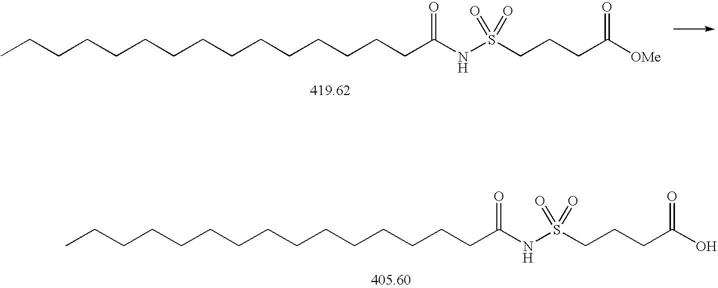

To a suspension of the methyl ester (0.84 g, 2.00 mmol) in methanol (10 ml) was added a solution of NaOH (0.54 g, 13.5 mmol) in water (1.0 ml). The mixture was stirred at room temperature for 4 h. A mixture of water (30 ml) and 1N HCl (20 ml) was added, and the product was isolated by filtration. Recrystallization from boiling MeCN (50 ml) yielded 0.64 g (79%) of the title compound as colorless plates. M.p.: 156-157° C.

¹H NMR (DMSO-d₆): δ 0.85 (m, 3H), 1.23 (br s, 24H), 1.49 (m, 2H), 1.85 (m, 2H), 2.25 (t, J=7 Hz, 2H), 2.39 (t, J=7 Hz, 2H), 3.38 (m, 2H), 11.15 (s, 1H).

### Example B

### Ac-His-Dab-Lys(hexadecanoyl)-c[Glu-Orn-D-Phe-Arg-Trp-Lys]-NH₂

Synthesized according to procedure analogous to that described above.

m/z: 753.8 (m2+2), RT:12.28 min.

### Example C

### Ac-His-Dab-Lys(hexadecanoyl)-c[Glu-Lys-D-Phe-Arg-Trp-Lys]-NH₂

Synthesized according to procedure analogous to that described above.

m/z: 760.7 (m2+2), RT:12.14 min.

### Example D

### Ac-His-Thr-Lys(hexadecanoyl)-c[Glu-Orn-D-Phe-Arg-Trp-Lys]-NH₂

Synthesized according to procedure analogous to that described above.

m/z: 754.3 (m2+2), RT:12.99 min.

### Example E

### Ac-His-Thr-Lys(hexadecanoyl)-c[G lu-Dab-D-Phe-Arg-Trp-Lys]-NH₂

Synthesized according to procedure analogous to that described above.

(m/z: 747.3 (m2+2), RT:13.10 min.

### Example F

### Ac-His-Thr-Lys(hexadecanoyl)-Gln-Lys-D-Phe-Arg-Trp-Nle-NH₂

Synthesized according to procedure similar to that described above.

m/z: 762.2 (m2+2), RT:13.6 min.

### Pharmacological Methods

Assay (I)—Experimental Protocol for Efficacy Testing on Appetite with MC4 Analogues, Using an Ad Libitum Fed Rat Model.

TAC:SPRD rats or Wistar rats from M&B Breeding and Research Centre A/S, Denmark are used for the experiments. The rats have a body weight of 200-250 g at the start of the experiment. The rats arrive at least 10-14 days before start of the experiment with a body weight of 180-200 g. Each dose of compound is tested in a group of 8 rats. A vehicle group of 8 rats is included in each set of testing.

When the animals arrive they are housed individually in a reversed light/dark phase (lights off 7:30 am, lights on 7:30 pm), meaning that lights are off during daytime and on during nighttime. Since rats normally initiate food intake when light is removed, and eat the major part of their daily food intake during the night, this set-up results in an alteration of the initiation time for food intake to 7:30 am, when lights are switched off. During the acclimatization period of 10-14 days, the rats have free access to food and water. During this period the animals are handled at least 3 times. The experiment is conducted in the rats' home cages. Immediately before dosing, the rats are randomised to the various treatment groups (n=8) by body weight. They are dosed according to body weight at between 7:00 am and 7:45 am, with a 1-3 mg/kg solution administered intraperitoneally (ip), orally (po) or subcutaneously (sc). The time of dosing is recorded for each group. After dosing, the rats are returned to their home cages, where they then have access to food and water. The food consumption is recorded individually every hour for 7 hours, and then after 24 h and sometimes 48 h. At the end of the experimental session, the animals are euthanised.

The individual data are recorded in Microsoft excel sheets. Outliers are excluded after applying the Grubbs statistical evaluation test for outliers, and the result is presented graphically using the GraphPad Prism program.

Assay (II)—Melanocortin Receptor 3 and 5 (MC3 and MC5) cAMP Functional Assay Using the AlphaScreen™ cAMP Detection Kit

The cAMP assays for MC3 and MC5 receptors are performed on cells (either HEK293 or BHK cells) stably expressing the MC3 and MC5 receptors, respectively. The receptors are cloned from cDNA by PCR and inserted into the pcDNA 3 expression vector. Stable clones are selected using 1 mg/ml G418.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer, and then resuspended in stimulation buffer to a final concentration of 1×10⁶ or 2×10⁶ cells/ml. 25 μl of cell suspension is added to the microtiter plates containing 25 μl of test compound or reference compound (all diluted in stimulation buffer). The plates are incubated for 30 minutes at room temperature (RT) on a plate-shaker set to a low rate of shaking. The reaction is stopped by adding 25 μl of acceptor beads with anti-cAMP, and 2 min later 50 μl of donor beads per well with biotinylated cAMP in a lysis buffer. The plates are then sealed with plastic, shaken for 30 minutes and allowed to stand overnight, after which they are counted in an Alpha™ microplate reader.

EC₅₀ values are calculated by non-linear regression analysis of dose/response curves (6 points minimum) using the Windows™ program GraphPad™ Prism (GraphPad™ Software, USA). All results are expressed in nM.

For measuring antagonistic activity in the MC3 functional cAMP assay, the MC3 receptors are stimulated with 3 nM α-MSH, and inhibited with increasing amounts of the potential antagonist. The IC₅₀ value for the antagonist is defined as the concentration that inhibits MC3 stimulation by 50%.

Assay (III)—Melanocortin Receptor 4 (MC4) cAMP Assay

BHK cells expressing the human MC4 receptor are stimulated with potential MC4 agonists, and the degree of stimulation of cAMP is measured using the Flash Plate® cAMP assay (NEN™ Life Science Products, cat. No. SMP004).

The human MC4 receptor-expressing BHK cells are produced by transfecting the cDNA encoding MC4 receptor into BHK570/KZ10-20-48, and selecting for stable clones expressing the MC4 receptor. The human MC4 receptor cDNA, as well as a CHO cell line expressing the MC4 receptor, may be purchased from Euroscreen™. The cells are grown in DMEM, 10% FCS, 1 mg/ml G418, 250 nM MTX and 1% penicillin/streptomycin.

Cells at approx. 80-90% confluence are washed 3× with PBS, lifted from the plates with Versene and diluted in PBS. They are then centrifuged for 2 min at 1300 rpm, and the supernatant removed. The cells are washed twice with stimulation buffer, and resuspended in stimulation buffer to a final concentration of 0.75×10⁶ cells/ml (consumption thereof: 7 ml per 96-well microtiter plate). 50 μl of cell suspension is added to the Flash Plate containing 50 μl of test compound or reference compound (all diluted in H₂O). The mixture is shaken for 5 minutes and then allowed to stand for 25 minutes at RT. The reaction is stopped by addition of 100 μl Detection Mix per well (Detection Mix=11 ml Detection Buffer+100 μl (˜2 μCi) cAMP [¹²⁵I] tracer). The plates are then sealed with plastic, shaken for 30 minutes, and allowed to stand overnight (or for 2 hours) and then counted in the Topcounter (2 min/well). In general, the assay procedure is as described in the Flash Plate kit-protocol (Flash Plate® cAMP assay (NEN™ Life Science Products, cat. No. SMP004)). However, the cAMP standards are diluted in 0.1% HSA and 0.005% Tween™ 20, and not in stimulation buffer.

EC₅₀ values are calculated by non-linear regression analysis of dose/response curves (6 points minimum) using the Windows™ program GraphPad™ Prism (GraphPad Software, USA). All results are expressed in nM.

### Assay (IV)—Melanocortin Receptor 1 (MC1) Binding Assay

The MC1 receptor binding assay is performed on BHK cell membranes stably expressing the MC1 receptor. The assay is performed in a total volume of 250 μl: 25 μl of ¹²⁵NDP-α-MSH (22 pM in final concentration), 25 μl of test compound/control and 200 μl of cell membrane (35 μg/ml). Test compounds are dissolved in DMSO. Radioactively labeled ligand, membranes and test compounds are diluted in buffer: 25 mM HEPES, pH 7.4, 0.1 mM CaCl₂, 1 mM MgSO₄, 1 mM EDTA, 0.1% HSA and 0.005% Tween™ 20. The samples are incubated at 30° C. for 90 min in Greiner microtiter plates, separated with GF/B filters that are pre-wetted for 60 min in 0.5% PEI, and washed 2-3 times with NaCl (0.9%) before separation of bound from unbound radiolabelled ligand by filtration. After filtration the filters are washed 10 times with ice-cold 0.9% NaCl. The filters are dried at 50° C. for 30 min, sealed, and 30 μl of Microscint 0 (Packard, cat. No. 6013616) is added to each well. The plates are counted in a Topcounter (1 min/well).

The data are analysed by non-linear regression analysis of binding curves, using the Windows™ program GraphPad™ Prism (GraphPad Software, USA).

### Assay (V)—Melanocortin Receptor 4 (MC4) Binding Assay

### In Vitro ¹²⁵NDP-α-MSH Binding to Recombinant BHK Cells Expressing Human MC4 Receptor (Filtration Assay).

The assay is performed in 5 ml minisorb vials (Sarstedt No. 55.526) or in 96-well filterplates (Millipore MADVN 6550), and using BHK cells expressing the human MC4 receptor (see Assay III, above). The BHK cells are kept at −80° C. until assay, and the assay is run directly on a dilution of this cell suspension, without further preparation. The suspension is diluted to give maximally 10% specific binding, i.e. to approx. 50-100 fold dilution. The assay is performed in a total volume of 200 μl: 50 μl of cell suspension, 50 μl of ¹²⁵NDP-α-MSH (≈79 μM in final concentration), 50 μl of test compound and 50 μl binding buffer (pH 7) mixed and incubated for 2 h at 25° C. [binding buffer: 25 mM HEPES (pH 7.0), 1 mM CaCl, 1 mM MgSO₄, 1 mM EGTA, 0.02% Bacitracin and 0.2% BSA]. Test compounds are dissolved in H₂O or DMSO and diluted in binding buffer. Radiolabelled ligand and membranes are diluted in binding buffer. The incubation is stopped by dilution with 5 ml ice-cold 0.9% NaCl, followed by rapid filtration through Whatman GF/C filters pre-treated for 1 hour with 0.5% PEI. The filters are washed with 3×5 ml ice-cold NaCl. The radioactivity retained on the filters is counted using a Cobra II auto gamma counter.

The data are analysed by non-linear regression analysis of binding curves, using the Windows™ program GraphPad™ Prism (GraphPad Software, USA).

### Assay (VI)—Evaluation of Energy Expenditure

TAC:SPRD rats or Wistar rats from M&B Breeding and Research Centre A/S, Denmark are used. After at least one week of acclimatization, rats are placed individually in metabolic chambers (Oxymax system, Columbus Instruments, Columbus, Ohio, USA; systems calibrated daily). During the measurements, animals have free access to water, but no food is provided to the chambers. Light:dark cycle is 12 h:12 h, with lights being switched on at 6:00. After the animals have spent approx. 2 hours in the chambers (i.e. when the baseline energy expenditure is reached), test compound or vehicle are administered (po, ip or sc), and recording is continued in order to establish the action time of the test compound. Data for each animal (oxygen consumption, carbon dioxide production and flow rate) are collected every 10-18 min for a total of 22 hours (2 hours of adaptation (baseline) and 20 hours of measurement). Correction for changes in O₂ and CO₂ content in the inflowing air is made in each 10-18 min cycle.

Data are calculated per metabolic weight [(kg body weight)^{0.75}] for oxygen consumption and carbon dioxide production, and per animal for heat. Oxygen consumption (VO₂) is regarded as the major energy expenditure parameter of interest.

1. A compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group, is either absent or represents C₁₋₄alkanoyl or R⁴, which is a protracting group, optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl, C₂₋₈alkenyl or C₂₋₈alkynyl;
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof.

2. A compound according to claim 1, wherein R⁴ represents a straight-chain, branched and/or cyclic C₈₋₂₂alkanoyl, C₈₋₂₂alkenoyl or C₈₋₂₂alkynoyl, all of which may optionally be substituted with one or more substituents selected from hydroxy, halogen, carboxyl and aryl, wherein said aryl may optionally be further substituted with one or more substituents selected from hydroxy, halogen and carboxyl;
or wherein R⁴ represents C₇₋₁₇alkyl-C(O)—NH—S(O)₂—(CH₂)₃—C(O)—, wherein said alkyl may be substituted with one or more halogens;
or wherein R⁴ represents R⁵—C(O)—NH—S(O)₂—(CH₂)₃—C(O)—, wherein R⁵ represents 1-(4-benzoyl-phenyl)ethyl;
or wherein R⁴ represents a steroidal group represented by formula II or IIa 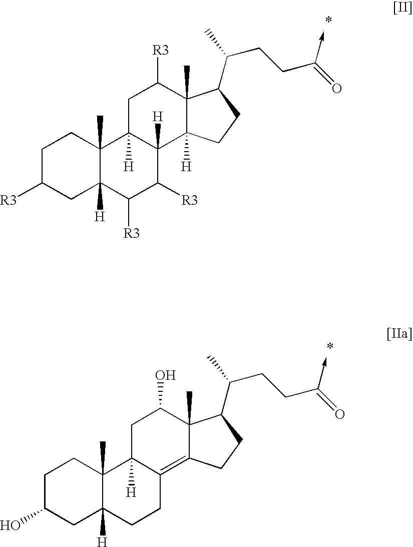
wherein each R³ independently represents hydrogen, hydroxy or (together with the bond via which R³ is attached to the ring carbon atom) ═O;
or wherein R⁴ represents a structure according to formula III, IIIa, IIIb, IV or IVa 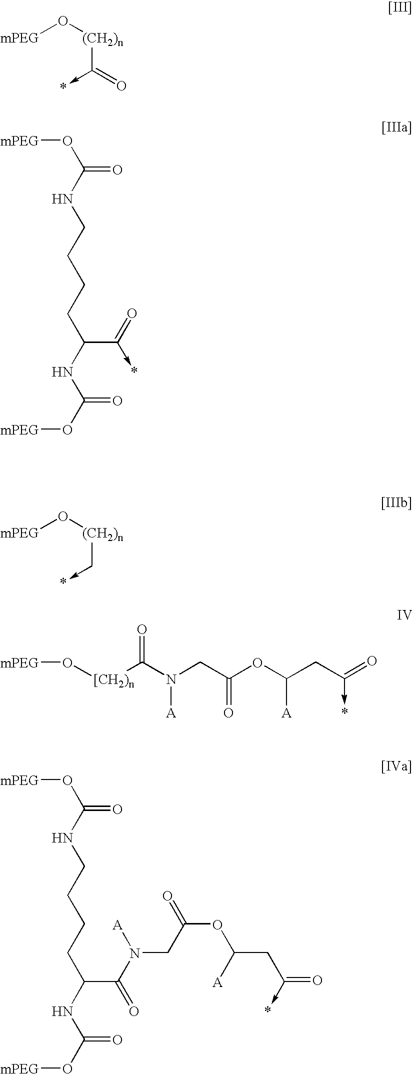
wherein n is 1, 2 or 3;
each mPEG independently represents methoxy-polyethyleneglycol with a molecular weight between about 2 kDa and about 50 kDa; and
each A independently represents hydrogen or C₁₋₄alkyl.

3. A compound according to claim 1, wherein said linker S, if present, represents β-Ala, Glu, Gly-Gln, Gly-Glu, Gly-His, or 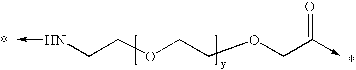
wherein y is 1, 2, 3, 4 or 5.

4. A compound according to claim 1, wherein X³ represents Lys, Orn, Dab, Dap, Cys, homoCys, Glu, Asp, Gln or Asn; and wherein X¹⁰ represents Lys, Orn, Dab, Dap, Cys, homoCys, Glu, Asp, Gln or Asp.

5. A compound according to claim 1, wherein there is a bridge between X³ and X¹⁰ rendering the compound of formula I cyclic, either by the presence of a disulfide bridge formed between X³ and X¹⁰ moieties independently selected from Cys and homoCys, or by the presence of a lactam bond formed between a carboxylic acid moiety in the side chain of X³ and an amine moiety in the side chain of X¹⁰, or between a carboxylic acid moiety in the side chain of X¹⁰ and an amine moiety in the side chain of X³.

6. A compound according to claim 4,
wherein X³ represents Glu or Asp; and
wherein X¹⁰ represents Lys, Orn, Dab or Dap.

7. A compound according to claim 4,
wherein X³ represents Glu or Asp; and
wherein X¹⁰ represents Lys.

8. A compound according to claim 1, wherein X⁵ represents Dab, Orn or Lys.

9. A compound according to claim 1, wherein R² represents —NH₂.

10. A compound according to claim 1, wherein X—X¹—X² represents
His-Dab-Lys(R⁴), His-Thr-Lys(R⁴), His-Dab-Lys(S—R⁴) or His-Thr-Lys(S—R⁴).

11. A compound according to claim 1, selected from the group consisting of:
Ac-His-Dab-Lys(4-(hexadecanoylsulfamoyl)butanoyl)-c[Glu-Dab-D-Phe-Arg-Trp-Lys]-NH₂,
Ac-His-Dab-Lys(hexadecanoyl)-c [Glu-Orn-D-Phe-Arg-Trp-Lys]-NH₂,
Ac-His-Dab-Lys(hexadecanoyl)-c [Glu-Lys-D-Phe-Arg-Trp-Lys]-NH₂,
Ac-His-Thr-Lys(hexadecanoyl)-c [Glu-Orn-D-Phe-Arg-Trp-Lys]-NH₂,
Ac-His-Thr-Lys(hexadecanoyl)-c[Glu-Dab-D-Phe-Arg-Trp-Lys]-NH₂ and
Ac-His-Thr-Lys(hexadecanoyl)-Gln-Lys-D-Phe-Arg-Trp-Nle-NH₂;
and pharmaceutically acceptable salts, prodrugs and solvates thereof.

12. A method of delaying the progression from IGT to type 2 diabetes, comprising administering to a patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group is either absent or represents C₁₋₄alkanoyl or R⁴ which is a protracting group optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue wherein the amino acid(s) may be naturally occurring or synthetic;
X_represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl, C₂₋₈alkenyl or C₂₋₈alkynyl;
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

13. A method of delaying the progression from type 2 diabetes to insulin-requiring diabetes, comprising administering to a patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group, is either absent or represents C₁₋₄alkanoyl or R⁴, which is a protracting group, optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl C₂₋₈alkenyl or C₂₋₈alkynyl
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

14. A method of treating obesity or preventing overweight, comprising administering to a patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group is either absent or represents C₁₋₄alkanoyl or R⁴, which is a protracting group optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl, C₂₋₈alkenyl or C₂₋₈alkynyl;
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

15. A method of regulating appetite, comprising administering to a patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group is either absent or represents C₁₋₄alkanoyl or R⁴, which is a protracting group optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl, C₂₋₈alkenyl or C₂₋₈alkynyl;
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

16. A method of inducing satiety, comprising administering to a patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group, is either absent or represents C₁₋₄alkanoyl or R⁴, which is a protracting group optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl C₂₋₈alkenyl or C₂₋₈alkynyl
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

17. A method of preventing weight gain after successfully having lost weight, comprising administering to a patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group, is either absent or represents C₁₋₄alkanoyl or R⁴, which is a protracting group optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl, C₂₋₈alkenyl or C₂₋₈alkynyl;
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

18. A method of increasing energy expenditure, comprising administering to a patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group, is either absent or represents C₁₋₄alkanoyl or R⁴, which is a protracting group, optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl C₂₋₈alkenyl or C₂₋₈alkynyl;
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

19. A method of treating a disease or state related to overweight or obesity, comprising administering to a patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group is either absent or represents C₁₋₄alkanoyl or R⁴, which is a protracting group optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl C₂₋₈alkenyl or C₂₋₈alkynyl;
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

20. A method of treating bulimia, comprising administering to a patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group, is either absent or represents C₁₋₄alkanoyl or R⁴, which is a protracting group optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl C₂₋₈alkenyl or C₂₋₈alkynyl
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

21. A method of treating binge-eating, comprising administering to a patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group, is either absent or represents C₁₋₄alkanoyl or R⁴, which is a protracting group optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl, C₂₋₈alkenyl or C₂₋₈alkynyl;
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

22. A method of treating a disease or state selected from atherosclerosis, hypertension, dia-betes, type 2 diabetes, impaired glucose tolerance (IGT), dyslipidemia, coronary heart dis-ease, gallbladder disease, gall stone, osteoarthritis, cancer, sexual dysfunction and risk of premature death, comprising administering to a patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group, is either absent or represents C₁₋₄alkanoyl or R⁴, which is a protracting group optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl C₂₋₈alkenyl or C₂₋₈alkynyl;
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

23. A method of treating, in an obese patient, a disease or state selected from type 2 diabetes, impaired glucose tolerance (IGT), dyslipidemia, coronary heart disease, gallbladder disease, gall stone, osteoarthritis, cancer, sexual dysfunction and risk of premature death, comprising administering to an obese patient in need thereof an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group, is either absent or represents C₁₋₄alkanoyl or R⁴, which is a protracting group, optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl C₂₋₈alkenyl or C₂₋₈alkynyl
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof, optionally in combination with one or more additional therapeutically active compounds.

24. A method according to claim 12, wherein said additional therapeutically active compound is selected from antidiabetic agents, antihyperlipidemic agents, antiobesity agents, antihypertensive agents and agents for the treatment of complications resulting from, or associated with, diabetes.

25. A method according to claim 24, wherein said compound is administered to said patient in a unit dosage form comprising from about 0.05 mg to about 1000 mg of said compound.

26. A method of activating MC4 in a subject, the method comprising administering to said subject an effective amount of a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group, is either absent or represents C₁₋₄alkanoyl or R⁴, which is a protracting group optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl, C₂₋₈alkenyl or C₂₋₈alkynyl
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof.

27. A method according to claim 12, wherein said compound is administered by parenteral or sublingual administration.

28. (canceled)

29. A pharmaceutical composition comprising a compound according to formula I:
R¹—X—X¹—X²—X³—X⁴—X⁵—X⁶—X⁷—X⁸—X⁹—X¹⁰—X¹¹—R² [I]
wherein R¹, which is bonded to an N-terminal NH₂-group, is either absent or represents C₁₋₄alkanoyl or R⁴, which is a protracting group optionally attached to X via a linker, S;
X represents a bond, or an amino acid residue, or a di- or tri-peptide residue, wherein the amino acid(s) may be naturally occurring or synthetic;
X¹ represents an amino acid residue with a functional group in the side chain to which a protracting group, R⁴, is attached, optionally via a linker, S;
X² represents a bond, or an amino acid residue, or a di-, tri- or tetra-peptide residue, wherein the amino acid(s) may be naturally occurring or synthetic;
X³ represents a bond, or an amino acid residue optionally capable of forming a bridge to X¹⁰;
X⁴ represents a bond, or an amino acid residue or di-peptide residue, wherein the amino acid(s) may be naturally occurring or synthetic;
X⁵ represents an amino acid residue selected from Dab, Dap, ornithine and lysine;
X⁶ represents D-Phe, wherein the phenyl moiety of D-Phe is optionally substituted with halogen, hydroxy, alkoxy, nitro, methyl, trifluoromethyl or cyano;
X⁷ represents Arg;
X⁸ represents Trp or 2-naphthylalanine;
X⁹ represents a bond, or an amino acid residue or di-peptide residue, wherein the amino acid(s) may be naturally occurring or synthetic;
X¹⁰ represents a bond, or an amino acid residue optionally capable of forming a bridge to X³;
X¹¹ represents a bond, or an amino acid residue or di-peptide residue, wherein the amino acid(s) may be naturally occurring or synthetic;
R² represents —OH or —NRR′, wherein R and R′ independently represent hydrogen, C₁₋₈alkyl, C₂₋₈alkenyl or C₂₋₈alkynyl;
wherein the compound of formula I is optionally cyclized from X³ to X¹⁰ via a lactam or a disulfide bridge;
with the proviso that the compound of formula I comprises at least 7 amino acid residues;
and pharmaceutically acceptable salts, prodrugs and solvates thereof.

30. (canceled)
